(19) United States
(12) Patent Application Publication
(10) Pub. No.: US 2024/0118259 A1
(43) Pub. Date: Apr. 11, 2024

(54) DEVICES AND SYSTEMS FOR MIMICKING HEART FUNCTION

(71) Applicant: UNIVERSITY OF CENTRAL FLORIDA RESEARCH FOUNDATION, INC., Orlando, FL (US)

(72) Inventors: James HICKMAN, Orlando, FL (US); Maria STANCESCU, Orlando, FL (US); Peter MOLNAR, Orlando, FL (US); Christopher LONG, Orlando, FL (US); Christopher McAleer, Orlando, FL (US)

(21) Appl. No.: 18/125,879

(22) Filed: Mar. 24, 2023

Related U.S. Application Data
(63) Continuation of application No. 16/913,528, filed on Jun. 26, 2020, now Pat. No. 11,614,437.
Application No. 16/913,528 is a continuation of application No. 14/764,683, filed on Jul. 30, 2015, now abandoned.
(60) Provisional application No. 61/758,628, filed on Jan. 30, 2013.
(60) Provisional application No. 61/790,061, filed on Mar. 15, 2013.
(60) Provisional application No. 61/758,628, filed on Jan. 30, 2013.
(60) Provisional application No. 61/790,061, filed on Mar. 15, 2013.

(51) Int. Cl. G01N 33/483; C12M 1/36; C12M 3/00

(52) U.S. Cl. CPC G01N 33/4836; C12M 21/08; C12M 41/48

(57) ABSTRACT

In an aspect, disclosed herein are physiological devices and systems, and components thereof, used to evaluate cardiac parameters and arrhythmogenic mechanisms. This abstract is intended as a scanning tool for purposes of searching in the particular art and is not intended to be limiting of the present invention.

### CROSS-REFERENCE TO RELATED APPLICATIONS

The present application claims the benefit of priority of U.S. Provisional Patent Application No. 61/758,628 filed Jan. 30, 2013 and of U.S. Provisional Patent Application No. 61/790,061 filed Mar. 15, 2013, each of which is incorporated herein by reference in its entirety.

### STATEMENT REGARDING FEDERALLY SPONSORED RESEARCH

This invention was made with government support under UH2-TR000516, R01-NS050452, and R01-EB005459 awarded by the National Institutes of Health. The United States government has certain rights in the invention.

### BACKGROUND OF THE INVENTION

The current drug development process is costly (e.g., approximately 1.2 billion dollars per drug) and time consuming (e.g., approximately 10-15 years per drug). Effective drug development to meet emergencies arising from pandemics or terrorism does not yet exist. There are several aspects of effective drug development. First, a drug screening device should identify key biomarkers and cellular responses that report physiological states. Second, to ensure accurate reporting of drug toxicology and efficacy, a device should provide physiologically relevant predictive modeling based on known clinical responses to drugs. Third, the cost should be bearable.

For example, cardiac main and side effects are major targets of pharmacological screening. The most commonly used screens with the highest predictive value are in vivo measurements on dogs, guinea pigs, or rabbits (De Clerck, 2002). However, these studies are low throughput, expensive, and suffer from interspecies differences (i.e., studies on conducted on non-humans). In most cases, animal research does not translate well to the human condition. However, the application of a high-throughput cardiac screen would save significant time and money. This would also eliminate drug failures in the clinical phase of drug development, thereby resulting in safer and cheaper drugs on the market. Therefore, a human-based in vitro system can provide the key technology necessary to speed up the drug discovery process by developing function-based human cell models that accurately capture and predict complex organ function.

These needs and other needs are satisfied by the present invention.

### BRIEF SUMMARY OF THE INVENTION

Disclosed herein are in vitro physiological systems comprising a microelectrode array; one or more cantilevers; cardiac myocytes; and a medium, wherein the system measures one or more cardiac parameters.

Disclosed herein are in vitro physiological systems comprising a microelectrode array; one or more cantilevers; cardiac myocytes; and a medium, wherein the system measures one or more arrhythmogenic mechanisms.

Disclosed herein are kits comprising an in vitro physiological system and instructions for using the in vitro physiological system to measure one or more cardiac parameters.

Disclosed herein are kits comprising an in vitro physiological system and instructions for using the in vitro physiological system to measure one or more arrhythmogenic mechanisms.

Disclosed herein are processes for synthesizing one or more components of a disclosed system, wherein the one or more components comprise cantilevers.

Disclosed herein are processes for synthesizing one or more components of a disclosed system, wherein the one or more components comprise microelectrode arrays.

Disclosed herein are methods of using a disclosed system.

Disclosed herein are methods of using a disclosed system to measure one or more cardiac parameters.

Disclosed herein are methods of using a disclosed system to measure one or more arrhythmogenic mechanisms.

Disclosed herein arc methods of using a disclosed system to recapitulate the electrical and contractile properties of a heart.

Disclosed herein are various uses for a disclosed in vitro physiological system.

Disclosed herein are uses of a disclosed system in one or more biomedical applications.

Disclosed herein are uses of a disclosed system in one or more toxicology studies

Disclosed herein arc uses of a disclosed system for drug screening.

Disclosed herein are uses of a disclosed system in lab-on-a-chip applications.

Disclosed herein are uses of a disclosed system in screening for individual medicines.

Disclosed herein are uses of a disclosed system for assessment or examination of genetic variances in the cells of a subject (e.g., induced adult cells iPSC).

### BRIEF DESCRIPTION OF THE SEVERAL VIEWS OF THE DRAWINGS

The accompanying Figures, which are incorporated in and constitute a part of this specification, illustrate several aspects of the invention and together with the description serve to explain the principles of the invention.

FIG. 1 shows a phase image of a monolayer of human cardiomyocytes cultured on a cantilever.

FIG. 2 shows a schematic representation of force measurement using an optical detection method.

FIG. 3A shows a representative voltage recording of human cardiomyocytes seeded on cantilevers before adding 3 μM of verapamil.

FIG. 3B shows a representative voltage recording of human cardiomyocytes seeded on cantilevers after adding 3 μM of verapamil.

FIG. 4A shows representative voltage changes detected in human cardiomyocytes seeded on cantilevers in extracellular solution containing 0 mM Ca²⁺.

FIG. 4B shows representative voltage changes detected in human cardiomyocytes seeded on cantilevers in extracellular solution containing 1 mM Ca²⁺.

FIG. 4C shows representative voltage changes detected in human cardiomyocytes seeded on cantilevers in extracellular solution after adding 1 μM norepinephrine.

FIG. 5A shows an image of the multichamber device on a rocker platform (e.g., a 10 organ chip system).

FIG. 5B shows an enlarged image of the multichamber device shown in FIG. 5A.

FIG. 6 shows an expanded view of a representative multichamber device, listing exemplary components of the device and exemplary tissues, cells, and/or organs for use in the device.

FIG. 7A shows how the disclosed system measures contractive force of patterned cardiomyocytes.

FIG. 7B shows how the disclosed system measures electrophysiological properties of patterned cardiomyocytes.

FIG. 7C shows a schematic of disclosed integrated system.

FIG. 8 shows the force generation (presented as contraction (uV) versus time (s)) of cardiomyocytes before (left panel) and after (right panel) addition of Ca²⁺.

FIG. 9 shows an image of a patterned microelectrode array (MEA).

FIG. 10A shows the effect of norepinephrine on the conduction velocity of human cardiomyocytes.

FIG. 10B shows the effect of norepinephrine on rhythm generation of human cardiomyocytes as compared to control.

FIG. 10C shows a bar graph representing frequency (Hz) versus concentration of norepinephrine.

FIG. 10D shows the effect of norepinephrine on QT interval of human cardiomyocytes.

FIG. 11A-FIG. 11C shows the effect of sotalol on various cardiac parameters such as QT interval and amplitude.

FIG. 12A shows a bar graph demonstrating % change versus concentration (μM) of sotalol.

FIG. 12B shows a bar graph demonstrating % change versus concentration (μM) of norepinephrine.

FIG. 12C shows a decrease in peak force versus concentration (μM) of human cardiomyocytes following administration of verapamil.

FIG. 13 shows the co-culture of liver cells (HepG2/C3A) and non-parenchymal cells within the RegeneMed scaffold inside the pumpless cell culture platform.

FIG. 14A shows examples of cell cultures on microfabricated membranes, specifically the fabrication of 3D membranes with macrovilli.

FIG. 14B shows that Caco-2 cells grown on SU-8 villi mimicked key aspects of the GI-tract epithelium.

FIG. 15A shows an image of an in vitro culture of cells after immortalization by knock-in of telomerase reverse transcriptasc (TERT).

FIG. 15B shows a magnified image of the image shown in FIG. 15A.

FIG. 15C shows an image of cultured cells stained for the stem cell marker Lgr5.

FIG. 15D shows a magnified image of the image shown in FIG. 15C.

FIG. 16A shows a fluorescent microscopy image of primary human colon epithelial cells stained for A33.

FIG. 16B shows a fluorescent microscopy image of primary human colon epithelial cells stained for cytokeratin 18.

FIG. 16C shows a fluorescent microscopy image of primary human colon epithelial cells stained for cytokeratin 20.

FIG. 16D shows a fluorescent microscopy image of primary human colon epithelial cells stained for villin.

FIG. 16E shows a fluorescent microscopy image of primary human colon epithelial cells stained for Muc2.

FIG. 16F shows a fluorescent microscopy image of primary human colon epithelial cells stained for chromogranin A.

FIG. 17A shows a feed-forward network as a component of a disclosed system used to examine the nervous system.

FIG. 17B shows an image of myotubes on cantilevers in a disclosed system used to examine the nervous system.

FIG. 17C shows a schematic of a disclosed system used to measure characteristics of cells in the nervous system.

FIG. 18A show an image of a neuron that displayed spine growth and extensive branching characteristic of mature neurons.

FIG. 18B shows immunocytochemical characterization of pyramidal cell using β-III tubulin.

FIG. 18C show the current flow of a cell that was cultured for 30 days in vitro.

FIG. 18D show the action potential generation of a cell that was cultured for 30 days in vitro.

FIG. 18E shows that a cell that was cultured for 30 days in vitro retained the ability to repeatedly fire.

FIG. 19 shows a composite image of a primary rat myotube co-cultured with primary rat motoneurons on a cantilever for 13 DIV and immunostained for Myosin Heavy Chain and β-III-Tubulin (cantilever edges were reinforced artificially to give an indication of their scale in relation to the cultured cells; scale bar=100 μm).

FIG. 20A shows a representative trace recording (in Volts) using laser deflection, indicating lengthwise strain on the cantilever from a myotube stimulated using broad field electrical pulses.

FIG. 20B shows the measurement of myotube contraction following neuronal stimulation via the addition of 200 μM glutamate.

FIG. 20C shows the measurement of myotube contraction following the addition of glutamate and 12.5 μM curare.

FIG. 21A shows an image of neuromuscular junction formation between human motoneurons and muscle derived from stem cells (40× magnification).

FIG. 21B shows an image of a potential synaptic site (arrow) in a day 15 co-culture demonstrated by co-localization of nerve terminals (indicated by synaptophysin) and AchR (indicated by BTX-488).

FIG. 22A shows an image of human cardiomyocytes 14 days in vitro (derived from human iPSCs, cultured in serum-free medium) obtained using light microscopy.

FIG. 22B shows an image of random (unpatterned) cardiomyocytes (derived from human iPSCs, cultured in serum-free medium) immunostained with troponin T and actin.

FIG. 22C shows an image of cardiomyocytes (derived from human iPSCs, cultured in serum-free medium) on patterned glass slides immunostained with troponin T and actin.

FIG. 22D shows an image of cardiomyocytes (derived from human iPSCs, cultured in serum-free medium) on patterned glass slides immunostained with troponin T and actin so as to demonstrate the directional alignment of muscle fibers.

FIG. 22E shows an enlarged image of the patterned cardiomyocytes on an MEA (derived from human iPSCs, cultured in serum-free medium).

FIG. 23 shows an image of cardiomyocytes (derived from human iPSCs) cultured on cantilevers for 19 days and immunostained for troponin T.

FIG. 24 shows a phase contrast micrograph of patterned cardiomyocytes 12 days in vitro (derived from human iPSCs) on top of substrate embedded extracellular electrodes.

FIG. 25A shows representative images of a disclosed device (both panels) (e.g., “hybrid systems laboratory” or “HSL”).

FIG. 25B shows a schematic of MEA chip (top panel) and a light micrograph of integrated cardiomyocytes 6 days in vitro (derived from human IPSCs) cultured on MEAs (bottom panel).

FIG. 25C shows a representative MEA recording of integrated cardiomyocytes 7 days in vitro using the device shown in FIG. 25A.

FIG. 25D shows a representative cantilever recording of integrated cardiomyocytes 7 days in vitro using the device shown in FIG. 25A.

FIG. 25E shows a representative image of a disclosed device comprising all the components of the device shown in FIG. 25A and a cantilever chip for measuring the contractile force of the cultured cardiomyocytes.

FIG. 26A shows the components of a representative disclosed device comprising both a cantilever chip for measuring contractile forces of cardiomyocytes and a MEA chip for measuring electrical activity of cardiomyocytes.

FIG. 26B shows an image of a cardiomyocytes cultured on an MEA for 2 days (top panel) and an image of cardiomyocytes cultured on cantilevers for 2 day (bottom panel).

FIG. 27 shows the field potential of spontaneously beating cardiomyocytes (derived from human iPSCs) cultured on an MEA.

FIG. 28 shows measurement of conduction velocity for cardiomyocytes (derived from human iPSCs) cultured on an MEA following electrical stimulation (at 500 mV, 2 Hz) propagated along the long loop pattern schematically represented to the right.

FIG. 29 shows the effect of sotalol on various cardiac parameters of cardiomyocytes (derived from human iPSCs) cultured for 12 days on an MEA.

FIG. 30A-FIG. 30D shows the response of cardiomyocytes cultured on silicon cantilevers comprising a DETA modification and fibronectin deposition as measured by contractile force (% change) following administration of norepinephrine (A), epinephrine (B), ouabain (C), and verapamil (D).

FIG. 31A shows a phase contrast image of human cardiomyocytes cultured in silicon cantilevers.

FIG. 31B shows a single action potential recording of cultured cardiomyocytes, which demonstrated a ventricular phenotype.

FIG. 31C shows that the beating rhythm and contractile force of cultured cardiomyocytes can be controlled with electrical stimulation (i.e., increasing stimulation frequencies from 0 Hz to 10 Hz increased beat frequency and decreased contractile forces).

FIG. 31D shows that cardiomyocytes cultured on cantilevers respond to norepinephrine by increasing contractile force and contractile frequency.

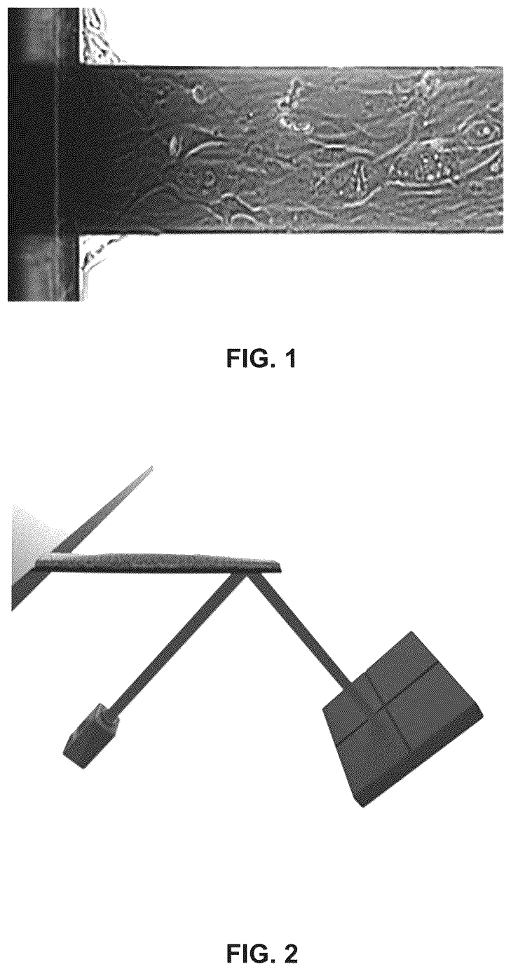

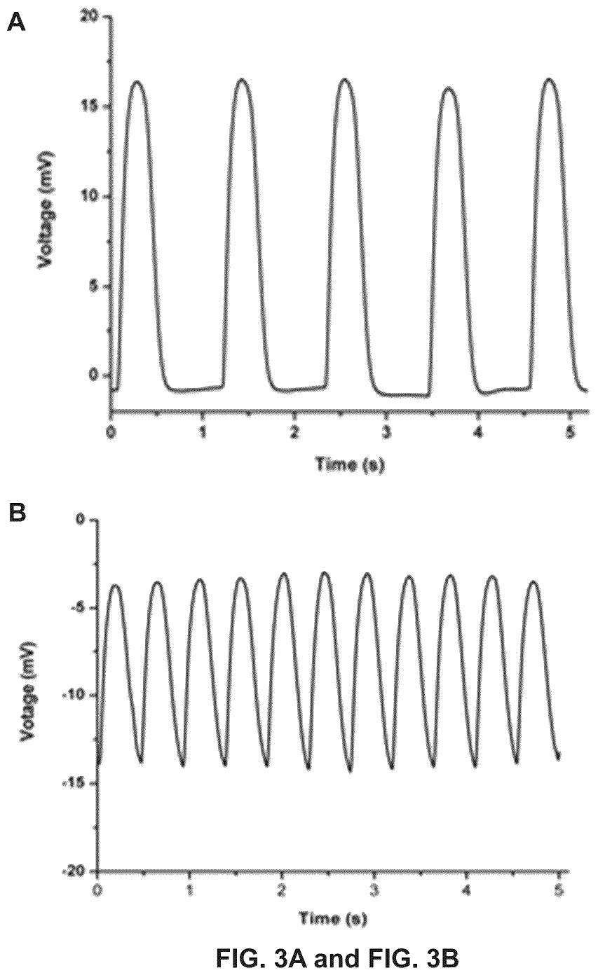

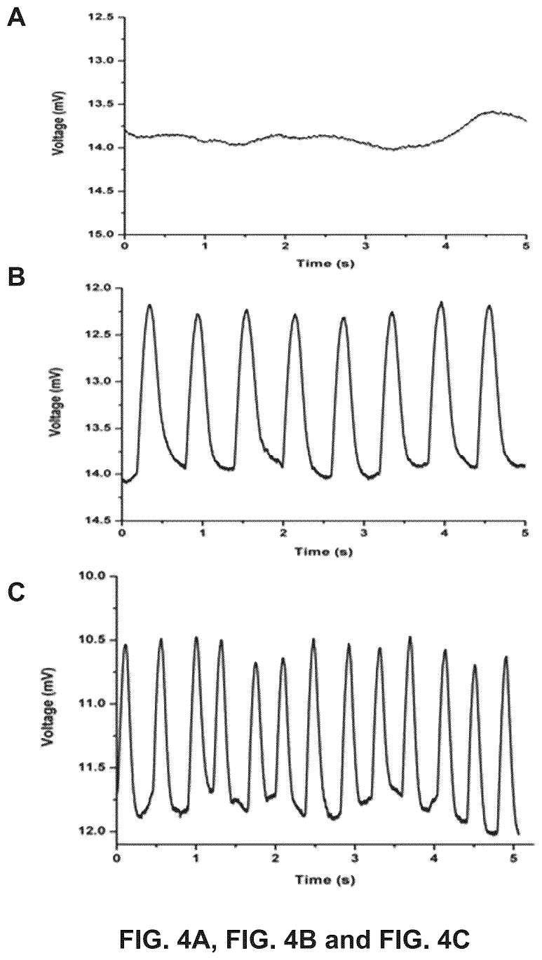

Additional advantages of the invention are set forth in part in the description that follows, and in part will be obvious from the description, or can be learned by practice of the invention. It is to be understood that both the foregoing general description and the following detailed description are exemplary and are not restrictive of the invention as claimed.

### DETAILED DESCRIPTION OF THE INVENTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Cardiac side effects are one of the major causes of failure for drug candidates in preclinical drug development or in clinical trials. Cardiac side effects are also responsible for the retraction of several marketed therapeutics. More than 850,000 people are hospitalized for arrhythmias each year and ventricular fibrillation (VF) is a leading cause of cardiac death (Jackson et al., 2004). Despite the intensive research in this area, the mechanism of VF is still poorly understood (Lin et al., 2008; Moe et al., 1962; Nash et al., 2006; and Pijnappels et al., 2007). Arrhythmia is a known side effect of commercial drugs. One of the mechanisms by which drugs can cause a potentially fatal form of ventricular tachy arrhythmia, called Torsades depointes (Tdp), is through the prolongation of the QT interval (in an ECG the length of the ventricular action potential). It has been reported that approximately 2-3% of all prescribed drugs can cause long QT syndrome (Recanatini et al., 2005; Sala et al., 2009). A broad range of cardiovascular drugs and antibiotics also have the potential risk of causing drug induced Tdp (Campbell et al., 2001; Hondeghem et al., 2007). At the same time, prolongation of the QT interval does not necessarily lead to Tdp; lengthening of the QT interval could even be anti-arrhythmogenic, as it is considered a mechanism of action of the class III anti-arrhythmics (Campbell et al., 2001; Hondeghem et al., 2007). Thus, a relatively high-throughput method to identify cardiac side effects and differentiate between arrhythmic and anti-arrhythmic effects at an early stage of drug development would have a significant impact on the field.

Gap junctions play an important role in the propagation of excitation in cardiac tissue. Changes in gap junction function affect major cardiac parameters, such as conduction velocity (CV). In several cardiovascular diseases, the expression of connexins (protein molecules that form gap junction channels) is decreased or their distribution is changed, leading to a malfunction in gap junction coupling. Understanding the pharmacological modulation of cardiac gap junction channels would further aid the drug development enterprise. The presently disclosed systems further aid this process.

### A. Definitions

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 arc disclosed, then 11, 12, 13, and 14 arc also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein, the terms “optional” or “optionally” can mean that the subsequently described event or circumstance can or cannot occur, and that the description includes instances where said event or circumstance occurs and instances where it does not. For example, in an aspect, in a method of using a disclosed system, the system can be optionally contacted with one or more second agents.

As used herein, the term “analog” can refer to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

As used herein, the term “subject” can refer to the target of administration, e.g., an animal. The term “subject” can include domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered.

As used herein, “cardiomyocytes” and “cardiac myocytes” can refer to the cells that constitute cardiac muscle. In an aspect, cardiomyocytes and cardiac myocytes can be used interchangeably.

As used herein, “cardiac output” can refer to the electrical rhythm generation and conduction system of the heart and/or by the force generation ability of the cardiac muscle. The skilled person in the art is familiar with “cardiac output” and the techniques available to measure cardiac output.

As used herein, the term “cardiac parameters” can refer to spontaneous beating rate, conduction velocity, field potential length (i.e., QT interval), (minimal inter-spike interval (i.e., shortest possible inter-spike interview), peak contractile force, speed of contraction, and/or time to relaxation. The skilled person in the art is familiar with “cardiac parameters” and the techniques available to measure cardiac parameters.

As used herein, “arrhythmogenic mechanisms” can refer to rhythm generation, conduction, and/or reentry. In an aspect, rhythm generation can comprise chronotropy and/or firing frequency dispersion. The skilled person in the art is familiar with “arrhythmogenic mechanisms” and the techniques available to measure arrhythmogenic mechanisms.

As used herein, “conduction” can refer to conduction velocity, conduction velocity dispersion, and/or frequency dependence of conduction velocity. The skilled person in the art is familiar with “conduction” and the techniques available to measure conduction.

As used herein, “reentry” can refer to QT interval, QT interval dispersion, reverse use dependence, absolute refractory period, and/or relative refractory period. The skilled person in the art is familiar with “reentry” and the techniques available to measure reentry.

A “patient” can refer to a subject afflicted with one or more diseases or disorders, such as, for example, a disease or disorder that affects the heart or affects cardiac muscle or affects cardiac parameters or arrhythmogcnic mechanisms.

As used herein, the term “treatment” can refer to the medical management of a patient with the intent to cure, ameliorate, stabilize, and/or prevent a disease, pathological condition, or disorder (such as, for example, a disorder that affects the heart). This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prevent” or “preventing” can refer to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” can mean having been subjected to a physical examination by a person of skill, for example, a physician, and found to have a condition that can be diagnosed or treated by the devices, systems, and methods disclosed herein. For example, in an aspect, a subject can be diagnosed with one or more conditions that affect the heart, heart muscle, and/or heart function, and/or cause abnormalities in the subject's various cardiac parameters.

The term “contacting” as used herein can refer to bringing a disclosed composition, compound, or complex together with an intended target (such as, e.g., a cell or population of cells, a receptor, an antigen, or other biological entity) in such a manner that the disclosed composition, compound, or complex can affect the activity of the intended target (such as, e.g., a cell or population of cells, a receptor, an antigen, or other biological entity.), either directly (i.e., by interacting with the target itself), or indirectly (i.e., by interacting with another molecule, co-factor, factor, or protein on which the activity of the target is dependent). In an aspect, one or more agents can be contacted with a disclosed system or disclosed device, or cells (e.g., cardiomyocytes) contained therein.

As used herein, the term “determining” can refer to measuring or ascertaining an activity or an event or a quantity or an amount or a change in expression and/or in activity level or in prevalence and/or incidence. Determining can refer to measuring one or more cardiac parameters. Determining can refer to measuring one or more arrhythmogenic mechanisms. In an aspect, measuring one or more cardiac parameters and/or one or more arrhythmogenic mechanisms can comprise a computer or a computer system. The skilled person in the art is familiar with the use of computers and computer systems (i.e., hardware, software, other equipment, etc.) to measure one or more cardiac parameters and/or one or more arrhythmogenic mechanisms among other things.

Methods and techniques used to determining an activity or an event or a quantity or an amount or a change in expression and/or in activity level or in prevalence and/or incidence as used herein can refer to the steps that the skilled person would take to measure or ascertain some quantifiable value. The art is familiar with the ways to measure an activity or an event or a quantity or an amount or a change in expression and/or in activity level or in prevalence and/or incidence

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. For example, in an aspect, an effective amount of a disclosed composition or complex is the amount effective to alter one or more cardiac parameters and/or one or more arrhythmogenic mechanisms in a desired cell or population of cells or organ such as the heart. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a disclosed composition or complex at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. In an aspect, a preparation can be administered in a “prophylactically effective amount”; that is, an amount effective for prevention of a disease or condition.

As used herein, “growth factors” can refer to proteins that bind to receptors on the surface of one or more cells to effect cellular proliferation and/or differentiation. Growth factors are known to the art and can include, but are not limited to, the following: Epidermal Growth Factor (EGF), Platelet-Derived Growth Factor (PDGF), Fibroblast Growth Factors (FGFs), Transforming Growth Factors-β TGFs-β), Transforming Growth Factor-α (TGF-α), Erythropoietin (Epo), Insulin-Like Growth Factor-1 (IGF-1), Insulin-Like Growth Factor-2 (IGF-2), Interleukin-1 (IL-1), Interleukin-2 (IL-2), Interleukin-6 (IL-6), Interleukin-8 (IL-8), Tumor Necrosis Factor-α (TNF-α), Tumor Necrosis Factor-β (TNF-β), Interferon-γ (INF-γ), and Colony Stimulating Factors (CSFs). In an aspect, one or more growth factors can be added to the serum-free medium.

As used herein, “hormone” can refer to a chemical that interacts with a receptor in a target tissue to effect a change in the function of that tissue. Hormones are known to the art and can include, but are not limited to, the following: Adrenocorticotrophic hormone, Antidiuretic hormone, Cortisol, Dehydroepiandrostendione, Dihydrotestosterone, Estrogens (e.g., estradiol, estrone, estriol), Follicle Stimulating hormone, Growth Hormone, Inhibin, Insulin, Luteinizing Hormone, Melanocyte Stimulating Hormone, Melatonin, Progesterone, Prolactin, Proopiomelanocortin, Testosterone, Thyroid Stimulating Hormone, Thyroxine, and Triiodothyronine.

As used herein, “ouabain” refers to a cardiac glycoside that inhibits ATP-dependent sodium-potassium exchange across cell membranes. The binding of ouabain to the sodium-potassium pump (also called Na⁺/K⁺ ATPase) prevents the conformational changes necessary for proper function. The structural formula for ouabain is presented below:

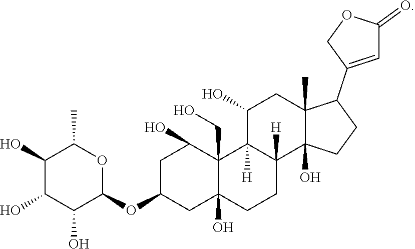

As used herein, “verapamil” or “verapamil hydrochloride” refers to a calcium ion influx inhibitor (slow-channel blocker or calcium ion antagonist) that exerts its pharmacologic effects by modulating the influx of ionic calcium across the cell membrane of the arterial smooth muscle as well as in conductile and contractile myocardial cells. The structural formula for verapamil is presented below:

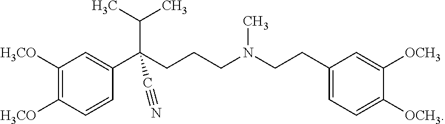 [SMILES: COc1ccc(CCN(C)CCC[C@@](C#N)(c2ccc(OC)c(OC)c2)C(C)C)cc1OC]

As used herein, “norepinephrine” refers to a precursor of epinephrine that is secreted by the adrenal medulla and is a widespread central and autonomic neurotransmitter. Norepinephrine is the principal transmitter of most postganglionic sympathetic fibers and of the diffuse projection system in the brain arising from the locus ceruleus. The structural formula of norepinephrine is presented below:

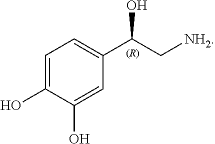

As used herein, “epinephrine” is a hormone and a neurotransmitter. Epinephrine is also referred to as adrenaline, adrenalin, or 4,5-β-trihydroxy-N-methylphenethylamine. Epinephrine is one of a group of monoamines called the catecholamines. The structural formula of epinephrine is presented below:

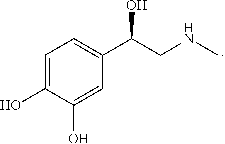

As used herein, “sotalol” or “sotalol hydrochloride” refers to an antiarrhythmic drug with Class II (beta-adrenoreceptor blocking) and Class III (cardiac action potential duration prolongation) properties. Chemically, sotalol hydrochloride is d,1-N-[4-[1-hydroxy-2-[(1-methylethyl) amino]ethyl]phenyl]methane-sulfonamide monohydrochloride. The structural formula of sotalol is presented below:

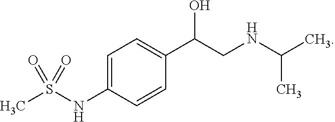 [SMILES: CC(C)NCC(O)c1ccc(NS(C)(=O)=O)cc1]

As used herein, the term “transducer” can refer to a device that converts one type of energy into another. This conversion can be to or from electrical, electromechanical, electromagnetic, photonic, photovoltaic, and other forms of energy. The skilled person in the art is familiar with transducers. For example, in an aspect, a disclosed transducer employs a piezoelectric microcantilever having a cardiac myocyte attached thereto. When the cardiac myocyte contracts, it can bend the microcantilever generating a piezoelectric signal. Conversely, if an electric signal is applied to the piezoelectric microcantilever, it can bend in response to the applied electric signal.

As used herein, the terms “cantilever” and “microcantilever” can be used interchangeably and can be used to identify the same component of a disclosed system.

As used herein, the term “piezoelectricity” can refer to the ability of certain materials (crystals and certain ceramics) to generate an electric potential in response to applied mechanical stress. The skilled person in the art is familiar with piezoelectric microcantilever fabrication and function.

### B. Systems

Disclosed herein are in vitro physiological systems comprising a microelectrode array, one or more cantilevers, cardiac myocytes, and a medium, wherein the system measures one or more cardiac parameters. Disclosed herein are in vitro physiological systems comprising a microelectrode array, one or more cantilevers, cardiac myocytes, and a medium, wherein the system measures one or more arrhythmogenic mechanisms. Disclosed herein are in vitro physiological systems comprising a microelectrode array, one or more cantilevers, cardiac myocytes, and a medium, wherein the system measures one or more cardiac parameters and one or more arrhythmogenic mechanisms. In an aspect, a disclosed system can represent a two-dimensional model of a human heart. In a disclosed system, cantilevers can be generated using a photolithographic process. A disclosed cantilever can comprise silicon. A disclosed cantilever can comprise a surface modification. Surface modifications are known to the skilled person in the art. In an aspect, a surface modification can comprise a coating of (3-Trimethoxysilylpropyl)diethylenetriamine. In an aspect, a cantilever can be contacted with fibronectin (i.e., fibronectin deposition).

In a disclosed system, microelectrode arrays can comprises glass. In an aspect, a disclosed microelectrode array can comprise one or more surface modifications. Surface modifications are known to the skilled person in the art. In the art, a surface modification can be generated using traditional protein absorption or can be generated using self-assembled monolayers (SAMs). In an aspect, SAMs comprise extracellular matrix components. Extracellular matrix components are known to the skilled person in the art and can comprise fibronectin, collagen, organo silanes containing amine moieties, and polyethylene-glycol moieties. Organo silanes containing amine moieties can comprise (3-Trimethoxysilylpropyl)diethylenetriamine. Polyethylene-glycol moieties can comprise 2-[Methoxy(polyethyleneoxy)propyl]trimethoxysilane. In an aspect, a disclosed microelectrode array can comprise patterning. Methods of patterning are known to the skilled person in the art. In an aspect, patterning on the microelectrode array can be generated using a 193 nm deep-UV excimer laser through a quartz photomask. In an aspect, a disclosed microelectrode array can be contacted with fibronectin.

In a disclosed system, cardiac myocytes can be human derived cardiac myocytes. In an aspect, human derived cardiac myocytes can be derived from differentiated human embryonic stem cells. In an aspect, human derived cardiac myocytes can be derived from adult induced pluripotent stem cells. In an aspect of a disclosed system, cardiac myocytes can be cultured on a microelectrode array. In an aspect, cardiac myocytes can be commercially purchased or can be cultured from a precursor cell type.

In a disclosed system, a medium can be serum-free. Mediums, including serum-free mediums, are known to the skilled person in the art. Methods and techniques to optimize one or more mediums for a particular cell type or a particular set of culture conditions are known to the skilled person in the art. For example, a disclosed medium can be optimized via the use of additional components, such as, for example, growth factors and/or hormones and/or antibiotics.

In an aspect, a medium of a disclosed system can comprise one or more growth factors. Growth factors are known to the art and include proteins that bind to receptors on the surface of one or more cells to effect cellular proliferation and/or differentiations. Growth factors can include, but are not limited to, the following: Epidermal Growth Factor (EGF), Platelet-Derived Growth Factor (PDGF), Fibroblast Growth Factors (FGFs), Transforming Growth Factors-β TGFs-β), Transforming Growth Factor-α (TGF-α), Erythropoietin (Epo), Insulin-Like Growth Factor-1 (IGF-1), Insulin-Like Growth Factor-2 (IGF-2), Interleukin-1 (1L-1), Interleukin-2 Interleukin-6 (1L-6), Interleukin-8 (1L-8), Tumor Necrosis Factor-α (TNF-α), Tumor Necrosis Factor-β (TNF-β), Interferon-γ (NF-γ), and Colony Stimulating Factors (CSFs). In an aspect, one or more growth factors can be added to the serum-free medium. In an aspect, a medium can comprise epidermal growth factor (EGF). The skilled person is familiar with various growth factors.

In an aspect, a medium of a disclosed system can comprise one or more hormones. Hormones can include, but are not limited to, the following: Adrenocorticotrophic hormone, Antidiuretic hormone, Cortisol, Dehydroepiandrostendione, Dihydrotestosterone, Estrogens (e.g., estradiol, estrone, estriol), Follicle Stimulating Hormone, Growth Hormone, Inhibin, Insulin, Luteinizing Hormone, Melanocyte stimulating hormone, Melatonin, Progesterone, Prolactin, Proopiomelanocortin, Testosterone, Thyroid Stimulating Hormone, Thyroxine, and Triiodothyronine. In an aspect, a disclosed medium can comprise L-thyroxin. In an aspect, a disclosed medium can comprise hydrocortisone. The skilled person is familiar with various hormones.

In an aspect, a disclosed system can measure contractile force of the cardiac myocytes. In an aspect, a disclosed system can measure electrical properties of the cardiac myocytes. In an aspect, a disclosed system can measure both contractile forcc and electrical properties of the cardiac myocytes.

One or more agents can be introduced into a disclosed system. Agents are known to the skilled person in the art. For example, agents include, but are not limited to, the following: metabolic inhibitors, nutritional supplements, therapeutic compounds, compositions, and drugs, investigational compounds, compositions, and drugs, biosimilars, agonists, antagonists, hormones, growth factors, small molecules, monoclonal antibodies, and combinations thereof.

In an aspect, one or more agents can be introduced to a disclosed system. In an aspect, following the introduction of one or more agents, a disclosed system can measure contractile force of the cardiac myocytes. In an aspect, following the introduction of one or more agents, a disclosed system can measure electrical properties of the cardiac myocytes. In an aspect, following the introduction of one or more agents, a disclosed system can measure both contractile force and electrical properties of the cardiac myocytes.

In an aspect, a disclosed system can evaluate arrhythmogenic mechanisms. In an aspect, following the introduction of one or more agents, a disclosed system can evaluate arrhythmogenic mechanisms. Arrhythmogenic mechanisms are known to the art and can comprise rhythm generation, conduction, and reentry. In an aspect, rhythm generation can comprise chronotropy and firing frequency dispersion. In an aspect, conduction can comprise conduction velocity, conduction velocity dispersion, and frequency dependence of conduction velocity. In an aspect, reentry can comprise QT interval, QT interval dispersion, reverse use dependence, absolute refractory period, and relative refractory period.

In an aspect, a disclosed system can evaluate parameters of cardiac function. In an aspect, following the introduction of one or more agents, a disclosed system can evaluate parameters of cardiac function. Parameters of cardiac function are known to the skilled person in the art. Parameters of cardiac function can comprise spontaneous beating rate, conduction velocity, QT interval, minimal inter-spike interval (upon high frequency stimulation), peak contractile force, speed of contraction, and time to relaxation. Thus, in an aspect, a disclosed system can evaluate one or more parameters of cardiac functions, wherein the one or more parameters comprise spontaneous beating rate, conduction velocity, QT interval, minimal inter-spike interval (upon high frequency stimulation), peak contractile force, speed of contraction, and time to relaxation.

In an aspect, a disclosed system can be used in conjunction with SCREENIT.

### C. Kits

Disclosed herein arc kits comprising an in vitro physiological system and instructions for using the in vitro physiological system to measure one or more cardiac parameters. Disclosed herein are kits comprising an in vitro physiological system and instructions for using the in vitro physiological system to measure one or more arrhythmogenic mechanisms. In a disclosed kit, an in vitro physiological system can comprise a microelectrode array, one or more cantilevers, cardiac myocytes, and a medium.

Cantilevers of a disclosed kit can be generated using a photolithographic process. A disclosed cantilever can comprise silicon. A disclosed cantilever can comprise a surface modification. Surface modifications are known to the skilled person in the art. In an aspect, a surface modification can comprise a coating of (3-Trimethoxysilylpropyl)diethylenetriamine. In an aspect, a cantilever can be contacted with fibronectin.

A microelectrode array of a disclosed kit can comprise glass. In an aspect, a disclosed microelectrode array can comprise one or more surface modifications. Surface modifications are known to the skilled person in the art. In the art, a surface modification can be generated using traditional protein absorption or can be generated using self-assembled monolayers (SAMs). In an aspect, SAMs comprise extracellular matrix components. Extracellular matrix components are known to the skilled person in the art and can comprise fibronectin, collagen, organo silanes containing amine moieties, and polyethylene-glycol moieties. Organo silanes containing amine moieties can comprise (3-Trimethoxysilylpropyl)diethylenetriamine.

Polyethylene-glycol moieties can comprise 2-[Metlioxy(polyethyleneoxy)propyl]trimetlioxysilane. In an aspect, a microelectrode array of a disclosed kit can comprise patterning. Methods of patterning are known to the skilled person in the art. In an aspect, patterning on the microelectrode array can be generated using a 193 nm deep-UV excimer laser through a quartz photomask. In an aspect, a disclosed microelectrode array can be contacted with fibronectin.

Cardiac myocytes of a disclosed kit can be human derived cardiac myocytes. In an aspect, human derived cardiac myocytes can be derived from differentiated human embryonic stem cells. In an aspect, human derived cardiac myocytes can be derived from adult induced pluripotent stem cells. In an aspect of a disclosed system, cardiac myocytes can be cultured on a microelectrode array. In an aspect, cardiac myocytes can be commercially purchased or can be cultured from a precursor cell type.

The medium of a disclosed kit can be serum-free. Mediums, including serum-free mediums, are known to the skilled person in the art. Methods and techniques to optimize one or more mediums for a particular cell type or a particular set of culture conditions are known to the skilled person in the art. For example, a medium of a disclosed kit can be optimized via the use of additional components, such as, for example, growth factors and/or hormones and/or antibiotics.

For example, in an aspect, a medium of a disclosed kit can comprise one or more growth factors. Growth factors are known to the art and include proteins that bind to receptors on the surface of one or more cells to effect cellular proliferation and/or differentiations. Growth factors can include, but are not limited to, the following: Epidermal Growth Factor (EGF), Platelet-Derived Growth Factor (PDGF), Fibroblast Growth Factors (FGFs), Transforming Growth Factors-β TGFs-(β), Transforming Growth Factor-α (TGF-α), Erythropoietin (Epo), Insulin-Like Growth Factor-1 (IGF-1), Insulin-Like Growth Factor-2 (IGF-2), Interleukin-1 (IL-1), Interleukin-2 (IL-2), Interleukin-6 (IL-6), Interleukin-8 (IL-8), Tumor Necrosis Factor-α (TNF-α), Tumor Necrosis Factor-β (TNF-β), Interferon-γ (INF-γ), and Colony Stimulating Factors (CSFs). In an aspect, one or more growth factors can be added to the serum-free medium. In an aspect, a medium can comprise epidermal growth factor (EGF).

For example, in an aspect, a medium of a disclosed kit can comprise one or more hormones. Hormones can include, but are not limited to, the following: Adrenocorticotrophic hormone, Antidiuretic Hormone, Cortisol, Dehydroepiandrostendione, Dihydrotestosterone, Estrogens (e.g., estradiol, estrone, estriol), Follicle Stimulating Hormone, Growth Hormone, Inhibin, Insulin, Luteinizing Hormone, Melanocyte Stimulating Hormone, Melatonin, Progesterone, Prolactin, Proopiomelanocortin, Testosterone, Thyroid Stimulating Hormone, Thyroxine, and Triiodothyronine. In an aspect, a medium of a disclosed kit can comprise L-thyroxin. In an aspect, a medium of a disclosed kit can comprise hydrocortisone.

In an aspect, components of a disclosed kit can be used measure contractile force of the cardiac myocytes. In an aspect, components of a disclosed kit can be used can measure electrical properties of the cardiac myocytes. In an aspect, components of a disclosed kit can be used to measure both contractile force and electrical properties of the cardiac myocytes.

One or more agents can be introduced into the in vitro physiological system of a disclosed kit Agents are known to the skilled person in the art. For example, agents include, but are not limited to, the following: metabolic inhibitors, nutritional supplements, therapeutic compounds, compositions, and drugs, investigational compounds, compositions, and drugs, biosimilars, agonists, antagonists, hormones, growth factors, small molecules, monoclonal antibodies, and combinations thereof.

In an aspect, one or more agents can be introduced to the in vitro physiological system of a disclosed kit. In an aspect, following the introduction of one or more agents, components of a disclosed kit can be used measure contractile force of the cardiac myocytes. In an aspect, following the introduction of one or more agents, components of a disclosed system can be used to measure electrical properties of the cardiac myocytes. In an aspect, following the introduction of one or more agents, components of a disclosed system can be used to measure both contractile force and electrical properties of the cardiac myocytes.

In an aspect, components of a disclosed kit can be used to evaluate arrhythmogenic mechanisms. In an aspect, following the introduction of one or more agents, components of a disclosed kit can be used to evaluate arrhythmogenic mechanisms. Arrhythmogenic mechanisms are known to the art and can comprise rhythm generation, conduction, and reentry. In an aspect, rhythm generation can comprise chronotropy and firing frequency dispersion. In an aspect, conduction can comprise conduction velocity, conduction velocity dispersion, and frequency dependence of conduction velocity. In an aspect, reentry can comprise QT interval, QT interval dispersion, reverse use dependence, absolute refractory period, and relative refractory period.

In an aspect, components of a disclosed kit can be used to evaluate parameters of cardiac function. In an aspect, following the introduction of one or more agents, components of a disclosed kit can be used to evaluate parameters of cardiac function. Parameters of cardiac function are known to the skilled person in the art. Parameters of cardiac function can comprise spontaneous beating rate, conduction velocity, QT interval, minimal inter-spike interval (upon high frequency stimulation), peak contractile force, speed of contraction, and time to relaxation. Thus, in an aspect, a disclosed kit can be used to evaluate one or more parameters of cardiac functions, wherein the one or more parameters comprise spontaneous beating rate, conduction velocity, QT interval, minimal inter-spike interval (upon high frequency stimulation), peak contractile force, speed of contraction, and time to relaxation.

In an aspect, a disclosed kit can be used in conjunction with SCREENIT.

### D. Methods Using the Disclosed Systems

Disclosed herein are methods of using a disclosed system. In an aspect, disclosed herein is a method of using a disclosed system to measure one or more cardiac parameters. In an aspect, disclosed herein is a method of using a disclosed system measure one or more arrhythmogenic mechanisms. Disclosed herein is a method of using a disclosed system to measure one or more cardiac parameters and to measure one or more arrhythmogenic mechanisms. In an aspect, disclosed herein is a method of using a disclosed system to recapitulate the electrical and contractile properties of a human heart.

### E. Uses of the Disclosed Systems

Disclosed herein are various uses for the disclosed in vitro physiological systems.

Disclosed herein are uses of a disclosed system in one or more biomedical applications. For example, disclosed herein is a use of a disclosed system in drug discovery. In an aspect, drug discovery can refer to discovery of drugs or agents or compounds that target cardiac output. In an aspect, cardiac output can be enhanced. In an aspect, cardiac output can be determined by the electrical rhythm generation and conduction system of the heart and by the force generation ability of the cardiac muscle. The general concept of drug discovery is known to the art.

Disclosed herein are uses of a disclosed system in one or more toxicology studies. In an aspect, toxicology studies can refer to the examination or assessment of drugs or agents or compounds that target cardiac output. In an aspect, cardiac output can be determined by the electrical rhythm generation and conduction system of the heart and by the force generation ability of the cardiac muscle. The general concept of toxicology studies is known to the art.

Disclosed herein are uses of a disclosed system for drug screening. The general concept of drug screening is known to the art. In an aspect, drug screening can refer to the screening of one or more drugs or agents or compounds for an ability to elicit cardiac effects and/or cardiac side effects.

Disclosed herein are uses of a disclosed system in lab-on-a-chip applications. Disclosed herein are uses of a disclosed system in screening for individual medicines. Disclosed herein are uses of a disclosed system for assessment or examination of genetic variances in the cells of a subject (e.g., induced pluripotent stem cells or iPSCs).

### F. Synthesis

Disclosed herein are processes for synthesizing one or more components of a disclosed system. For example, processes for synthesizing a patterned microelectrode array are described in Natarajan et al., 2011, which is incorporated herein by reference in its entirety for its teachings regarding the synthesis of a patterned microelectrode array. For example, processes for synthesizing a silicon cantilever are described in Wilson et al., 2010, which is incorporated herein by reference in its entirety for its teachings regarding the synthesis of a silicon cantilever.

It is contemplated that each disclosed method can further comprise additional steps, manipulations, and/or components. It is also contemplated that any one or more step, manipulation, and/or component can be optionally omitted. It is understood that a disclosed methods can be used to provide the disclosed compounds. It is also understood that the products of the disclosed methods can be employed in the disclosed methods of using.

### G. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and arc not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention. Efforts have been made to ensure accuracy with respect to numbers (e.g., amounts, temperature, etc.), but some errors and deviations should be accounted for.

The disclosed systems are based on human cardiac myocytes, though the invention contemplates cardiac myocytes from subjects not limited to humans, for example, subjects listed herein, including but not limited to animals. As described herein, the incorporation of a functional cardiac system based on patterned cardiac cells integrated with microelectrode assays enabled the measurement of several cardiac parameters including conduction velocity, peak amplitude and spontaneous frequency, QT interval, and relative refractory period (which is related to triangulation). In doing so, the disclosed systems demonstrated high predictive value for cardiac side effects, electrical conduction, and cardiac muscle force generation.

### Materials and Devices

### a. Preparation of Cell Culture Medium

In the disclosed system, the human-derived cardiac myocytes were cultured in a serum-free medium. The medium was optimized to enhance human cardiac myocyte growth and differentiation. Specifically, the medium was supplemented with specific growth factor such as epidermal growth factor (EGF) or hormones such as hydrocortizone and L-thyroxin. In an aspect, the serum-free medium can comprise 100 mL Ultraculture medium (Bio Whittaker Cambrex) supplemented with 10 mL B27, 1 mL L-glutamine (Gibco/Invitrogen), 1 mL Penicillin Streptomycin, 0.375 g dextrose (Fisher Scientific) in 800 μL water, 1 mL non-essential amino acids and 1 mL of Hepes buffer (Gibco/Invitrogen) (Sathaye et al., 2006). Additional growth factors can also added to improve cell survival in the serum-free conditions including 0.1 μg/mL of L-thyroxine, 10 ng/mL of epidermal growth factor (Sigma-Aldrich), and 0.5 μg/mL of hydrocortisone (BD biosciences).

### b. Fabrication of Cantilever

The layout for the cantilevers was generated using AutoCAD 2004. The patterns were written to chrome coated 5-inch sodalime glass masks for front and backside photolithography. Cantilevers were fabricated from 6-inch double-sided polished silicon-on-insulator (SOI) wafers with a 5μm crystalline silicon layer (front side) and a 500 lum silicon dioxide layer (back side). The front side was primed with a 10 nm layer of hexamethyldisilazane (HMDS) to promote resist adhesion. A 5 μm layer of the photoresist AZ 5214 E (Clariant, Muttenz, Switzerland) was spun onto the device layer followed by softbake, alignment, exposure, and development. The device layer was etched using the deep reactive ion etch (DRIE) process at a rate of 2 μm/min. Resist was stripped and a 0.5 μm thick layer of silicon dioxide was deposited via Plasma Enhanced Chemical Vapor Deposition (PECVD) to protect the device layer during subsequent processing. The wafer was then flipped over and was primed with a 10 nm layer of HMDS and spun with 4.15 μm layer of AZ 9245 photoresist (Clariant, Muttenz, Switzerland). Coating was followed by softbake, front-back alignment, development, and DRIE etch at 4 μm/min until the bulk of the back side had been etched through leaving only the buried native oxide layer. The devices were then subjected to a buffered HF dip to remove the buried native oxide layer as well as the silicon dioxide that had been deposited onto the device layer. Individual devices were separated by breaking connecting tabs that were incorporated into the device design. Cantilever dimensions were measured using a JEOL 6400 scanning electron microscope (SEM) at a take-off angle of 50° off normal.

The silicon cantilevers were coated with the amine-terminated alkylsilane, (3-Trimethoxysilylpropyl)diethylenetriamine (United Chemical Technologies, Bristol, PA) to promote cell adhesion and differentiation (Ravenscroft et al., 1998). Cantilevers were cleaned in serial acid baths of concentrated HC1 in methanol (1:1 dilution) for 30 minutes and concentrated H₂SO₄ for 1 hour, followed by 30 minutes in boiling de-ionized water. Cleaned cantilevers were dried overnight in an 80° C. oven. Surface modification was performed according to a previously published protocol (Das et al., 2008). Briefly, the cantilevers were incubated in 0.1% solution of (3-Trimethoxysilylpropyl)diethylenetriamine in toluene for 30 minutes under gentle heating (approximately 70° C.), followed by 3× wash in fresh toluene. The coated cantilevers were then heated in fresh toluene for 30 minutes followed by drying overnight in an 80° C. oven. Coated samples were stored in a desiccator until use. X-ray photoelectron spectroscopy (XPS) and contact angle measurements were used to characterize the surface coating.

### C. Fabrication of Piezoelectric Elements for Cantilevers

The skilled person in the art is familiar with piezoelectric microcantilever fabrication and function (see, e.g., Choudhury et al., 2007 and Datar et al., 2009). Piezoelectricity is the ability of certain materials (crystals and certain ceramics) to generate an electric potential in response to applied mechanical stress (Holler et al., 2007). The piezoelectric effect is used in various sensors to measure stresses or geometrical deformations in various mechanical devices. The reverse piezoelectric effect turns piezoelectric material into actuators, when an external voltage is applied to the crystal (King et al., 2000). Piezoelectric materials are known to the art and include, but are not limited to, the following: quartz, bone, sodium tungstate, zinc oxide, or lead zirconate titanate (PZT) (Lou 2009). A similar effect is the piezoresistive phenomenon. When subjected to mechanical stress, these materials change their resistivity (Mutyala et al., 2009).

Silicon wafers with silicon on insulator can serve as base material in the fabrication of piezoelectric cantilevers. An additional layer of 100-200 nm SiO₂ can be deposited onto the base material to insulate conductive materials from the semi-conductive silicon. Metal layers can be fabricated to connect the piezoelectric components with microelectronics. Layers of piezoelectric materials, such as ZnO and PTZ sol-gel, can be deposited exactly in those areas where microcantilevers remain after the etching process. Another conductive layer contacts the piezoelectric components from top to apply voltages for actuation or current read out during sensor mode. An insulation layer of silicon-ONO-stacks (oxide-nitride-oxide) can protect conductive elements from aqueous solutions during cell culture. Alternatively, piezoelectric elements can be replaced by piezoresistive materials. This alternative approach can offer a higher sensitivity during readout; however, piezoresistive materials do not provide the usage of the cantilevers as actuators and a field stimulator would be needed.

### d. Fabrication of Microelectrode Array

Generally, the amount of information that could be obtained using traditional MEAs was limited. Not only contact interaction with the surface, but also the shape of the attachment area determines the physiology of cardiac myocytes. Pattern geometries determine the extent of the alignment of the long axis of cardiac myocytes, alignment determines CV and other physiological and pharmacological properties of cardiac tissues (Parker et al., 2008; Bourgeious et al., 2009; Badie et al., 2009). Therefore, in the experiments described herein, the cardiac myocytes were patterned on the top of the microelectrodes. In doing so, the measurement of conduction velocity along a well defined path became possible, which extended the capability of MEA measurements by including field potential (AP) length. Consequently, the amount of information that could be obtained using MEAs increased. As described herein, the incorporation of a functional cardiac system based on patterned cardiac cells integrated with MEAs enabled the measurement of conduction velocity, peak amplitude and spontaneous frequency, QT interval and relative refractory period (which is related to triangulation).

Cardiac myocytes cultured on microelectrode arrays (MEA) have several benefits compared to either traditional patch clamp electrophysiology or isolated organ methods. The use of MEAs in the investigation of cardiac side effects is more cost effective when compared to standard patch-clamp electrophysiology. Furthermore, cardiac myocytes can be maintained over longer periods of time on MEAs, thus chronic experiments can be feasible. Furthermore, in the system disclosed herein, the serum-free medium used in to culture the cardiac myocytes increases the reproducibility of the disclosed system.

Cardiac myocytes can be patterned on glass using photolithography following surface modification with self-assembled monolayers (SAMs) for myocytes (Dhir et al., 2009, Molnar et al., 2007). The benefit of this method is the compatibility of the technique with cheap automated silicon manufacturing steps and the ability of the cells to self-assemble after random plating. Self-assembled monolayers or SAMs are one molecule thick monolayers attached to a surface composed of organic molecules. Surface modification with SAMs is also compatible with advanced photolithography methods (Ravesncroft et al., 1998; Corey et al., 1991). Cells survive on patterned surfaces for extended periods of time (Das et al., 2008; Das et al., 2010), do not migrate off the patterned areas (Corey et al., 1991) and exhibit the typical morphology and physiology of the specific cell type (Das et al., 2004 Lochter et al., 1995).

MEA's containing sixty (60) electrodes (10 μm diameter) (Multichannel Systems, Germany) were cleaned by soaking the arrays in a detergent solution for 2 hours followed by sonication for 10 minutes. The arrays were then oxygen plasma cleaned for 20 minutes. Surface modification was completed by incubation of the MEAs in a 3 mM PEG silane, 2-[Methoxypoly(ethyleneoxy)propyl]trimethoxysilane (MW=460-590, Gelest), solution in toluene, with 37% concentrated HCl added to achieve a final value of 0.08% (0.8 mL HCl/L), for 45 minutes at room temperature. The arrays were then rinsed once in toluene, twice in ethanol, twice in water and sonicated in water for 2 minutes to remove the non-covalently linked material (Popat et al., 2004). The arrays were air dried with nitrogen and stored in a dessicator overnight.

The MEAs were patterned using a deep UV (193 nm) excimer laser (Lambda Physik) at a pulse power of 230 mW and a frequency of 10 Hz for 45 seconds through a quartz photomask (Bandwidth foundry, Eveleigh, Australia). The arrays were sterilized using 70% isopropanol and then incubated with 5 μg/mL of fibronectin in a phosphate buffered solution (Invitrogen) for 20 minutes at room temperature. The solution was removed and the surface was first rinsed with PBS, followed by the plating medium, and then dried before the cells were plated.

SAM-modified surfaces arc characterized using XPS to demonstrate formation of the SAM and contact angle measurements to quantify wettability. Contact angle measurements are a rapid and simple measure of wettability. Contact angles are measured by application of static, sessile drops (5-30 μL) of deionized water to substrate surfaces with a micropipetter. The measurements are made visually on both sides of the drops using a Rame-Hart type goniometer. XPS is a technique for the elemental analysis and characterization of surfaces (Briggs 1992). Since the electrons of each clement possess characteristic binding energies, the energy pattern of emitted photoelectrons arising from a given element serves to unambiguously identify that element, while the precise peak positions, or chemical shifts, reflect the chemical environment (i.e., oxidation state) in which the element is found. XPS measurements are obtained on a FISONS 220i XL spectrometer. For each sample examined by XPS, a survey spectrum and high-resolution spectra for the elements Si, C, N, and any other clement that is unique to the SAM (F for 13F) was obtained. These measurements serve as (a) baseline quantities against which to contrast properties of the surface after cell culture, and (b) baseline quantities against which to contrast cell growth and survival from experiment to experiment for multivariate analysis.

### e. Detection System Setup

Spontaneous or electrical stimulation-evoked force generation of cardiac myocytes was measured based on optical detection or electrical detection of cantilever-bending. Optical detection of displacement of the end of the cantilever was based on the principle routinely used in atomic force microscopes (AFM) in which a laser beam was reflected from the cantilever to a sensor. The sensor detected the displacement of the laser beam caused by changes in the position of the cantilever. In this optical detection method, an automated electrical shutter was placed in the beam path. This placement minimized the exposure of the cells to the reflected laser light. Electrical detection of the displacement was based on piezoelectric principle that requires special design and materials for the fabrication of the cantilevers. Both detection methods enabled automation and high-throughput screening on myocyte contraction force. Peak contraction force and force-contraction velocity relationships were calculated based on the geometry of the cantilevers and the thickness of cultured cardiac myocytes (determined from immunostaining data). Compounds (such as norepinephrine, epinephrine, ouabain, verapamil, and sotalol) were applied using traditional methods to the medium and possible changes in force or force-velocity relationship were detected and analyzed using standard statistical methods.

A detection system similar to those used in atomic force microscope (AFM) system was designed for measuring deflection of the cantilevers during contraction. The entire system was assembled around an upright Olympus BX51WI electrophysiology microscope (Olympus Inc., Center Valley, PA). The detection system consisted of a class 2 red photodiode laser (Newport, Irvine, CA), a stimulation chamber, a 4-quadrant photodetector (Noah Industries, Melbourne, FL), and a computer with pClamp 10.0 data acquisition software (Molecular Devices, Union City, CA). The laser and photodetector (PD) were mounted on x-y-z-h translators (Newport, Irvine, CA), which were mounted on the underside of the microscope stage. The stimulation chamber was fabricated from 5 mm thick polycarbonate sheet. An approximately 15 mm×15 mm square chamber was milled out of the sheet and fitted with silver wires (0.015 inch diameter) for field stimulation. The silver wires were mounted parallel to each other with a separation of 15 mm. The bottom of the chamber was sealed using a 22 mm×22 mm glass coverslip. This created a transparent base through which the laser beam could easily pass. The silver wires were connected to an external pulse generator (A-M systems, Sequim, WA) capable of producing field stimulation pulses of varying intensity, frequency, and waveform. Both the pulse generator and PD were connected to an Axon Instruments series 1440 digitizer (Molecular Devices, Union City, CA) which was interfaced with the computer.

The AFM system was calibrated using a modified version of the optical lever method. A bare microcantilever die, without cells, was placed in the stimulation chamber. The laser was focused on one of the microcantilevers and the PD was adjusted so that the laser fell on the diode surface. Using a digital volt meter to monitor the output voltage, the PD was adjusted so that the voltage being read was less than −7 volts. The PD was then moved vertically in 5 mm increments and the voltage recorded at each position. The results were plotted in Excel and a linear regression line was fitted to the linear region of the calibration curve, which was the region between −5 and 5 volts. The slope of this region was the detector sensitivity (ydetector). This value was used to calculate the angle, 0, of the deflection at the end of the microcantilever using the equation (Equation 1):

$$θ = \frac{{y}_{measured}}{2 cos ( φ ) l \times {y}_{detector}} \qquad ( 1 )$$

where, y measured is the voltage measured from the PD, φ, is the angle of the detector to normal, and l, is the path length of the reflected laser beam.

Because of the large variability in the spring constants, cantilevers have to be further calibrated on an individual basis when used for precision force measurements. This variability is most likely caused by variations in thickness of the cantilever. Variability in the length and width is often quite small because typical lateral resolution in photolithography is on the submicrometer scale. For nominal spring constants greater than 0.1 N/m, the calibrated load displacement transducer of a nanoindenter can be used to measure the spring constant of each cantilever in an array. Measuring the resonance frequency of individual cantilevers and applying it to Sader's equation can provide detailed spring constants.

### f. Multielectrode Extracellular Recordings

The cardiac myocytes were cultured on patterned metal MEAs (planar 10 μm electrodes, 200 μm separation, Multichannel-systems). A 60 channel amplifier (MEA1040, Multichannel-systems) was used to record electrical activity from the spontaneously beating cardiac cells. The same electrodes were also used for stimulation utilizing a stimulus generator (STG 1002, Multichannel systems). The cells were stimulated utilizing 500 mV, 1 ms wide bipolar pulses at 2 Hz. The recording medium was the same as the plating medium with the pH adjusted to 7.3 using HEPES buffer. After a 30 minute incubation period, APs were detected and recorded using built in functions of the Multichannel System software. For drug experiments, 50 μM 1-Heptanol (Gibco/Invitrogen) was added to the bathing medium and recordings were performed before and 15 minutes after drug administration with additional recordings done at 15 minute intervals. For Sparfloxacin (Sigma-aldrich), 2 μM of the drug was added to the recording medium and recordings were taken in 15 minute intervals before and after drug administration. The data was further analyzed using software written using Matlab and Clampfit (Axon instruments).

### g. Cell Culture

The serum-free medium comprised 100 mL Ultraculture medium (Bio Whittaker Cambrex) supplemented with 10 mL B27, 1 mL L-glutamine (Gibco/Invitrogen), 1 mL Penicillin Streptomycin, 0.375 g dextrose (Fisher Scientific) in 800 μL water, 1 mL non-essential amino acids and 1 mL of Hepes buffer (Gibco/Invitrogen) (Sathaye et al., 2006). Additional growth factors were also added to improve cell survival in the serum-free conditions. They included 0.1 μg/mL of L-thyroxinc, 10 ng/mL of epidermal growth factor (Sigma-Aldrich) and 0.5 ng/mL of hydrocortisone (BD biosciences). Cells were plated at a density of 1000 cells/mm² on the MEAs. The medium was changed 24 hours after plating. Subsequent changing of the medium was performed every third day.

Human embryonic stem cell derived cell types including cardiomyocytes have been generated for pharmacology testing and toxicology screening. For example, GE markets cardiac myocytes. Before product release, each lot is highly characterized by flow cytometry, subcellular imaging, and electrophysiology. Each lot is demonstrated to express Gata4, Nkx2.5, MYH6/7, troponin I, aMHC and a-actinin, and negative for Oct4, TRA-1-81 and TRA-1-60. The cardiomyocyte population comprises ventricular, atrial, and nodal subtypes and has been documented to have an adult phenotype.

### h. Immunostaining

Patterned cardiac myocytes can be immunostained. In a set of experiments, the patterned cardiac myocytes were immunostained for F-Actin with Rhodamine Phalloidin (Invitrogen, R415) using a protocol provided by the company. Briefly, the cells were washed with PBS and fixed using 3% Formaldehyde. The coverslips were extracted with 0.1 mL Triton X® . The staining solution (with 1% Bovine Serum Albumin to prevent background staining) was added at a dilution of 1:40 in PBS and coverslips were incubated for 30 minutes. Imaging was done using confocal microscopy.

### ii) Experiment 1

In the experiments described herein, the integration of human cardiac myocytes into the two devices was achieved. The surface of the glass microelectrode arrays (MEAs) were coated with polyethylene-glycol (PEG) self-assembled monolayers (SAMs), which were then patterned with a 193 nm deep-UV excimer laser through a quartz photomask. The MEAs were incubated with fibronectin to create an attachment surface for the cardiac myocytes. The silicon cantilevers were treated with fibronectin. Human embryonic stem cell derived cardiac myocytes, which were obtained from a commercial source (GE), formed a monolayer on the surface of the two devices. The human derived cardiac myocytes displayed adult-like characteristics as verified by immunohistochemistry and electrophysiological and pharmacological experiments. Here, the cells differentiated to spontaneously contracting cardiac myocytes and showed stable activity for longer than a month.

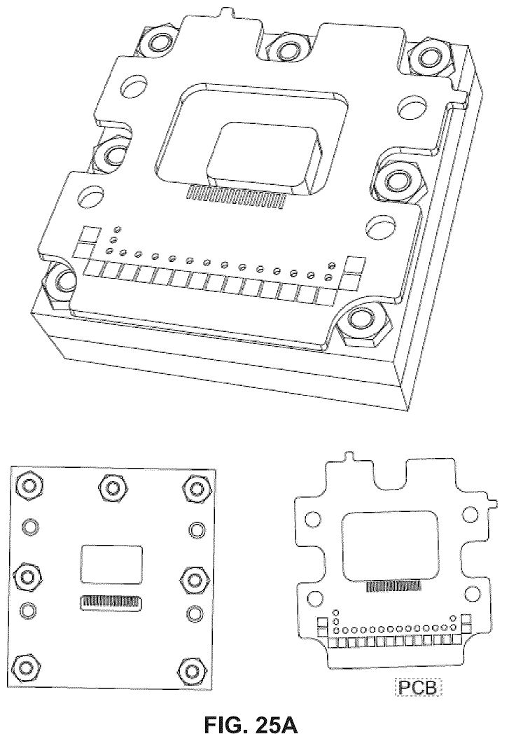

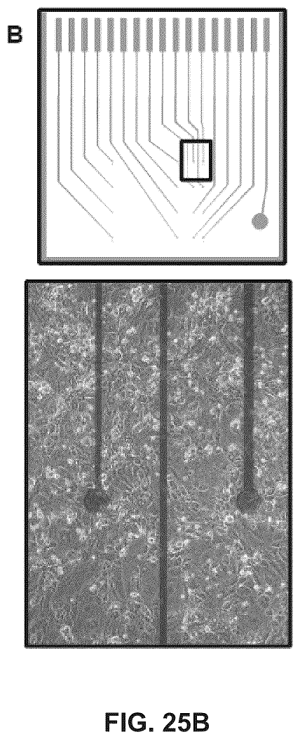

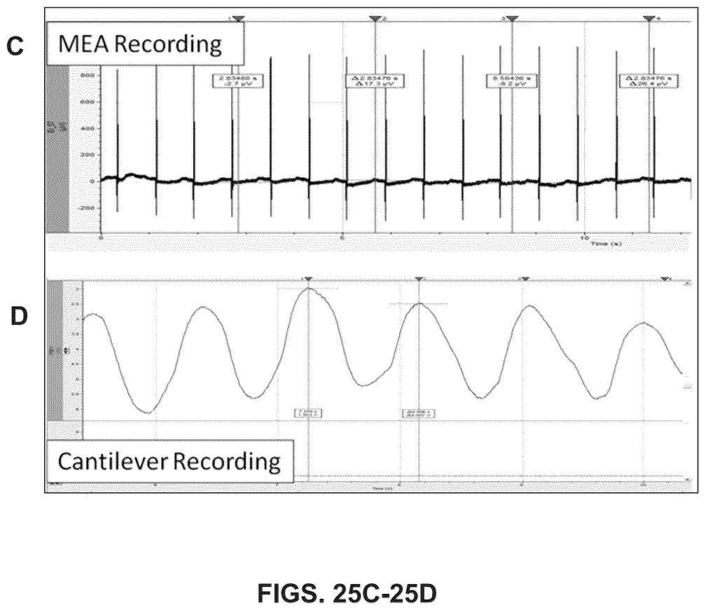

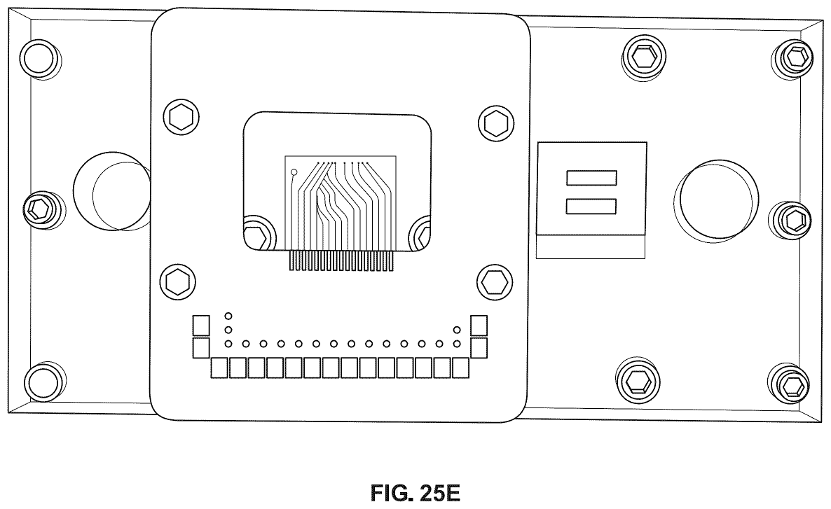

For example, as shown in FIG. 25A, a disclosed device (which can also be referred to as a “hybrid systems laboratory” or “HSL”) was comprised of the following components: a chip (a microelectrode array chip), a polycarbonate housing (bottom and top plates), two silicone gaskets, a printed circuit board (PCB), and a commercially available elastomeric connector. In FIG. 25A, the chip was made of fused silica and measured 15 mm×15 mm. Seventeen (17) titanium/platinum microelectrodes were microfabricated on the surface of the chip for measurements of the electrical activity of the cardiomyocytes plated on the chip surface. The MEA chip was placed between the two polycarbonate plates that were clamped together with screws. The silicone gaskets, placed between the polycarbonate plates and the cMEA chip, ensured tight scaling of the module. Two apertures were provided on the top polycarbonate plate. One aperture was used to access the portion of surface of the chip in which cardiomyocytes were plated and to perform medium changes. The other aperture provided access the contact pads of the chip microelectrodes. The PCB was placed on the top of the housing. Electrical contact between the cMEA chip and the PCB was made via an elastomeric connector placed in the aperture on the polycarbonate plate. Recording of the cardiomyocytes electrical activity were performed connecting the PCB to the Microelectrode Array System (Multichannel System). Data generated by integrated cardiomyocytes using the device are shown in FIG. 25C (field potential recordings via MEA) and FIG. 25D (force contraction recordings via cantilevers).

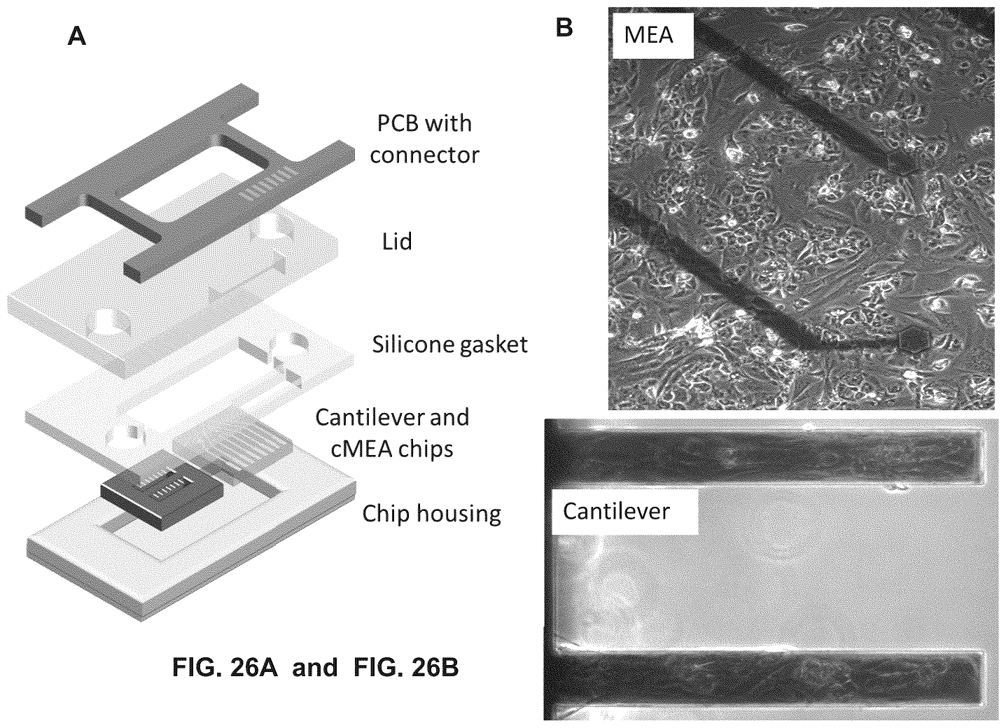

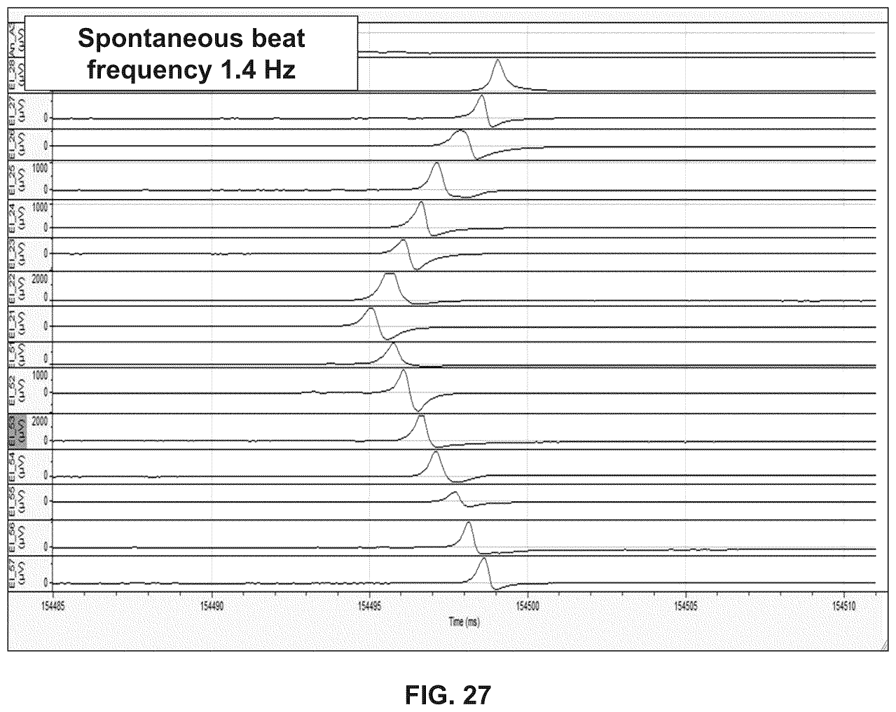

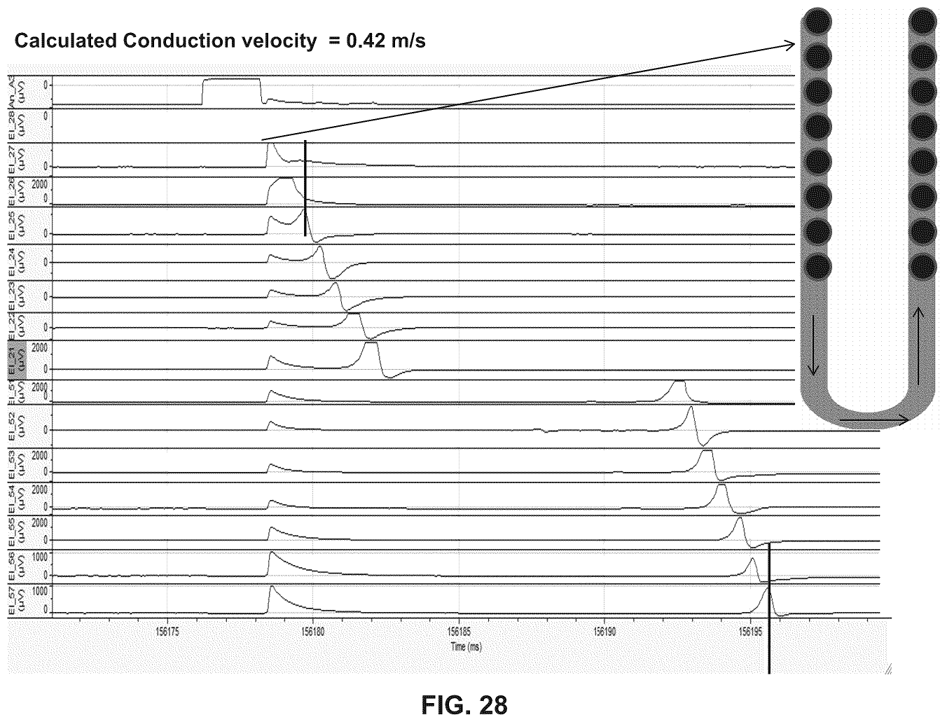

FIG. 25E shows an expanded version of the HSL in which the device comprises a cantilever chip for measuring the contractile forces of the cultured cardiomyocytes (see also, schematic shown in FIG. 26A). The cardiomyocytes used in these experiments were derived from differentiated adult induced pluripotent stem cells purchased from Cellular Dynamics. in FIG. 27 and FIG. 28, the electrical activity of the human cardiomyocytes plated on patterned MEAs was recorded following 12 days in vitro using a Multichannel Systems 60 channel amplifier (MEA 1040, Multichannel Systems). Prior to recording, the cells were allowed to equilibrate for 15 min in the lab atmosphere at 37° C. Temperature was maintained with a TC02 temperature controller (Multichannel Systems). The cells were stimulated using a STG 1002 stimulator (Multichannel Systems) by applying 1 ms wide bipolar square pulses of 500 mV every 500 ms. The recording medium was the same as the plating medium. In FIG. 28, the electrical stimulus was applied on the electrode located at the end of the loop (channel 28). Conduction velocity was calculated as distance divided by the time difference between the recorded peaks.

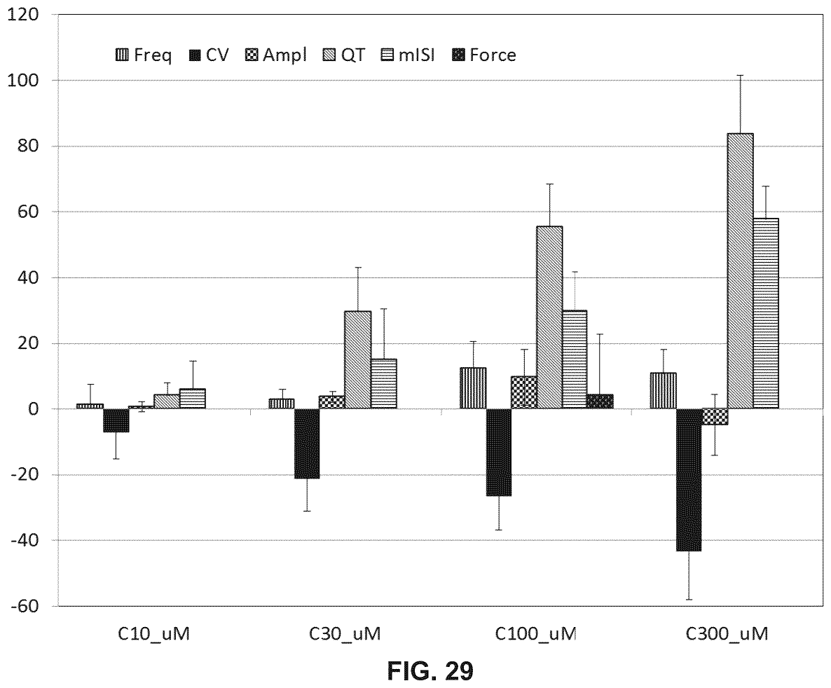

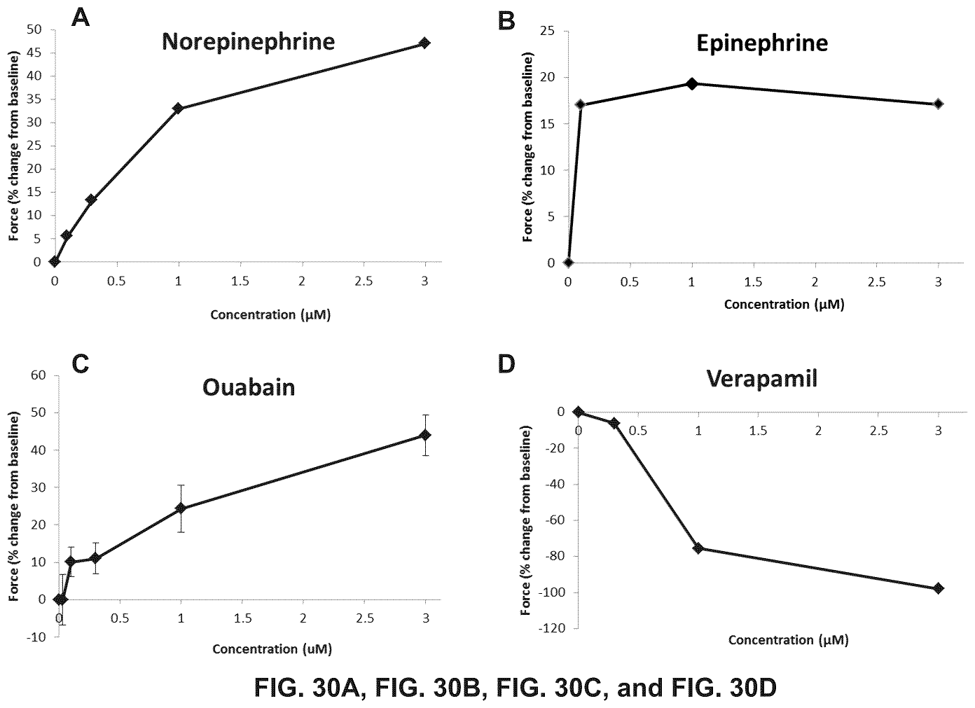

A disclosed system can be used to assess or evaluate the effects on cardiac parameters and/or cardiac outcomes generated by at least, and without limitation, the following: metabolic inhibitors, nutritional supplements, therapeutic compounds, compositions, and drugs, investigational compounds, compositions, and drugs, biosimilars, agonists, antagonists, hormoncs, growth factors, small molecules, monoclonal antibodies, and combinations thereof. For example, in the examples discussed below, several compounds were selected for use in the disclosed system. These compounds were selected, in part, because of an ability to affect specific cardiac parameters (See, for example, FIG. 29 and FIG. 30A-30D).

In the disclosed system, sotalol significantly increased QT intervals and mISI parameters. However, sotalol concentrations above 100 μM concentrations decreased conduction velocity. Thus, sotalol affected the measured cardiac parameters in a unique and characteristic way (i.e., created a “fingerprints”) according to its mechanism of action and physiological effects. For example, in FIG. 29, several cardiac parameters were measured including frequency (Freq), conduction velocity (CV), field potential amplitude (Ampl), QT interval, minimum interspike interval (mISI), and peak contraction force. Data are represented as percentage changes compared to the control and expressed as mcan+/−SEM. In FIG. 29, electrical activity of the human cardiomyocytes plated on patterned MEAs was recorded following 12 days in vitro (DIV) using a Multichannel Systems 60 channel amplifier (MEA 1040, Multichannel Systems). Prior to recording, the cells were allowed to equilibrate for 15 min in the lab atmosphere at 37° C. Temperature was maintained with a TC02 temperature controller (Multichannel Systems). The cells were stimulated using a STG 1002 stimulator (Multichannel Systems) by applying 1 ms wide bipolar square pulses of 500 mV every 500 ms. The recording medium was the same as the plating medium. Sotalol (Sigma, cat#S0278) was added to the bathing medium in increasing concentrations of 0 μM, 10 μM, 30 μM, 100 μM, and 300 μM.

In the disclosed system, norepinephrine significantly increased spontaneous beating rate, decreased QT interval (and it analogue minimal ISI), and increased peak contraction force of human cardiac myocytes in the concentration range of 0.1-3 μM. Thus, norepinephrine affected the measured cardiac parameters in a unique and characteristic way (i.e., created a “fingerprints”) according to its mechanism of action and physiological effects (FIG. 30A).

In the disclosed system, verapamil concentration dependently decreased contraction peak force. Thus, verapamil affected the measured cardiac parameters in a unique and characteristic way (i.e., created a “fingerprints”) according to its mechanism of action and physiological effects (FIG. 30D).

In FIG. 30A-FIG. 30D, which also shows the effect of ouabain (C) and epinephrine (B) on contractile force, the contractile stress of human cardiomyocytes plated on cantilevers was recorded following 14 day in vitro. A detection system similar to that used in atomic force microscopy (AFM) was used in these experiments. Prior to recording, the cells were allowed to equilibrate for 15 min in the lab atmosphere at 37° C. Drugs were added cumulatively as follow: 0.1 μM, 0.3 μM, 1.0 μM, and 3.0 μM (norepinephrine); 0.1 μM, 1.0 μM, and 3.0 μM (epinephrine); 0.1 μM, 0.3 μM, 1.0 μM, and 3.0 μM (ouabain); and 0.3 μM, 1.0 μM, and 3.0 μM (verapamil). Washout was performed by rinsing the cells five (5) times with 1 mL of cell culture medium.

Thus, the effects on action potential (AP) length (analog to the QT interval on the EEG recordings) and AP shape can be used in in vivo and ex vivo cardiac screens to predict arrhythmogenic effects of drugs.

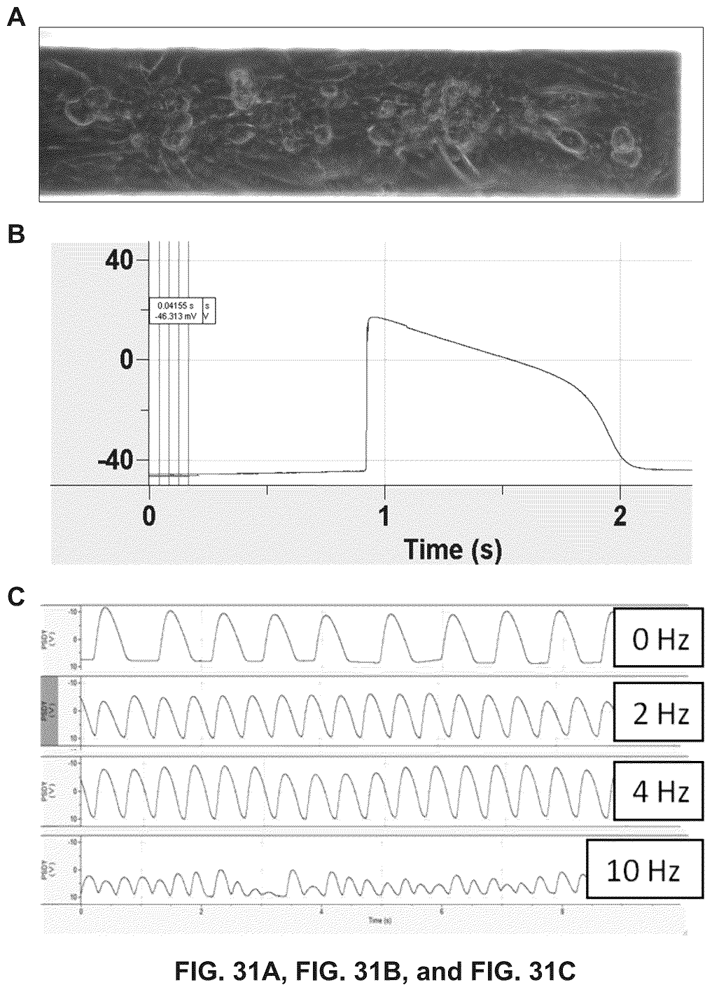

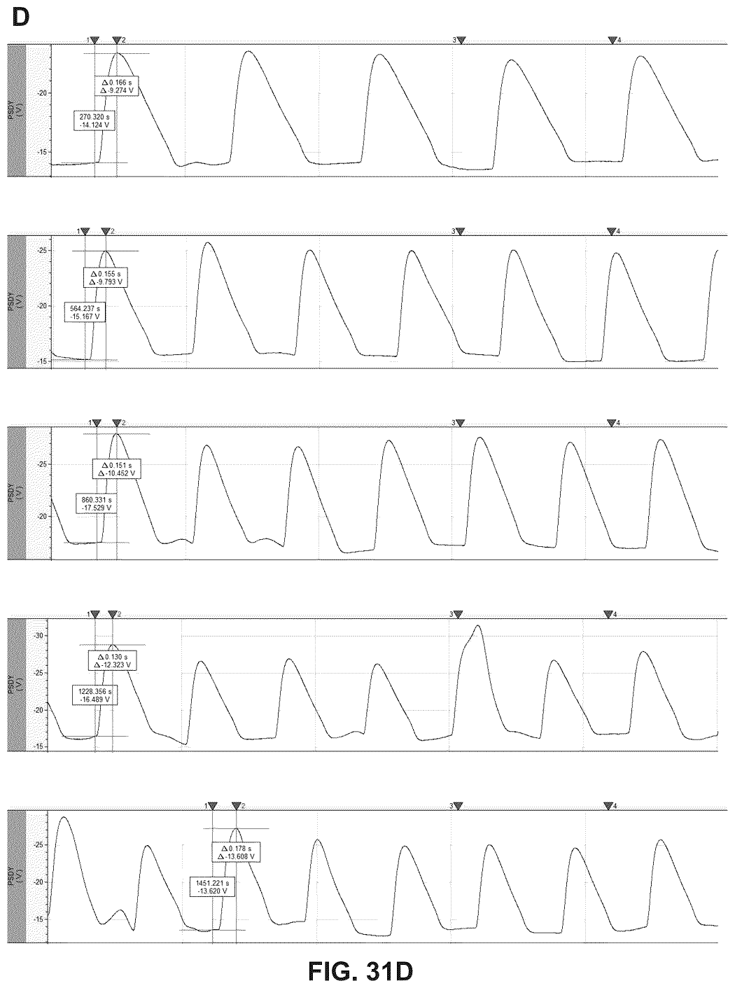

FIG. 31A-FIG. 31D provide data relating to the functional characterization of cardiomyocytes derived from human iPSCs cells. In FIG. 31B, whole-cell patch clamp recordings were performed with an Axioscope FS2 upright microscope (Carl Zeiss, Gottingen, Germany). Cells were perfused with extracellular solution containing NaCl 140 mM, KCl 5 mM, MgCl₂ 1 mM, CaCl₂ 2 mM, D-Glucose 10 mM, and HEPES 10 mM. The pH was adjusted to 7.4 and the osmolarity was 330 mOsm. The intracellular solution was composed KCl 140 mM, NaCl 4 mM CaCl₂ 0.5 mM, MgCl₂ 1 mM, EGTA 1 mM HEPES 10 mM, and Na₂ATP 5 mM. Patch pipettes were prepared from borosilicate glass (BF150-86-10; Sutter, Novato, CA) with a Sutter P97 pipette puller. Pipette resistance was 4-6 MOhm for intracellular patch clamp recordings. Voltage clamp and current clamp recordings were performed with a Multiclamp 700B amplifier (Axon Instruments, Foster City, CA, USA). Signals were filtered at 2 kHz and digitized at 20 kHz with an Axon Digidata 1322A interface.

For stimulation experiments shown in FIG. 31C square electrical stimuli, 40 ms wide, 4-5 V in amplitude were applied with varying frequencies ranging from 0 to 10 Hz via an isolated pulse stimulator (A-M Systems, Sequim, WA). Prior to recording, the cells were allowed to equilibrate for 15 min in the lab atmosphere at a temperature of 37° C. maintained by a Delta T4 culture dish temperature controller (Bioptechs, Butler, PA).

### iii) Experiment 2

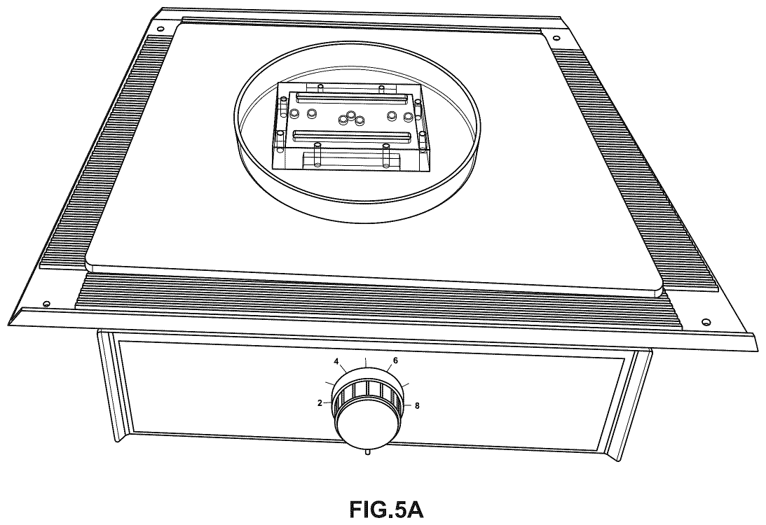

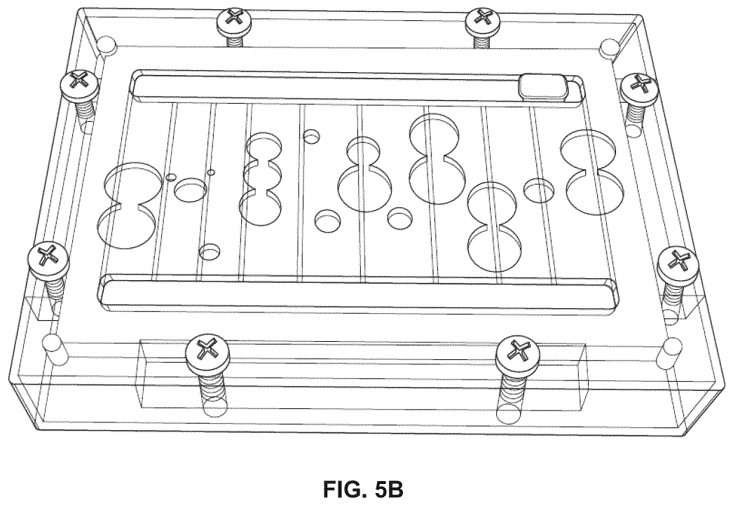

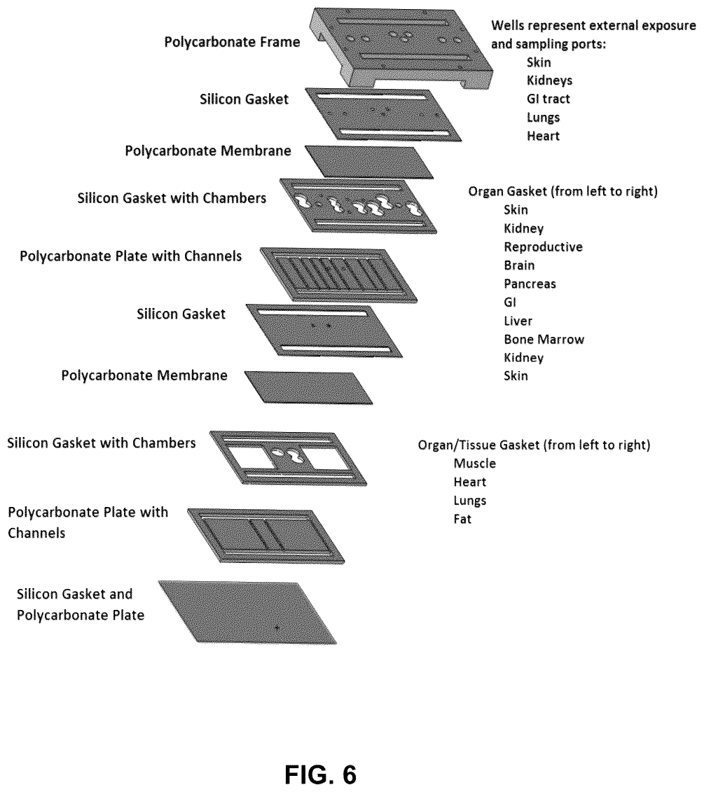

A low cost, easy to use platform to emulate human response to drugs and chemicals with 10 organ systems was constructed. The platform is “pumpless” and uses a rocker platform and gravity for the fluid motion force (FIG. 5A-5B). Since many units can be placed on a rocker platform and platforms can be stacked (FIG. 6), the system can be used for moderately high throughput studies. Studies have demonstrated that a platform can be constructed that is operational and can sustain viable cell cultures of HepG2/C3A cells in all compartments for at least 48 hours. Studies with dyes and without cells demonstrate appropriate transfer across polymeric membranes in compartments (e.g., the blood brain barrier). The mixing time to achieve near equilibrium in the system corresponds to physiologic values. The initial characterization studies indicate that such a platform system is feasible.

The “pumpless” cell culture platform is a multichamber device on a rocker platform made from silicone sheets and a polycarbonate frame (see, e.g., FIG. 5A, FIG. 5B, and FIG. 6). It is produced at a low cost and is easy to modify, easy to implement (rapid set-up and minimal operator training). The low cost format is due to no pump, multiple units on a rocker platform, and optical and electrical access. The platform has a robust operation that includes no gas bubbles, removes tubing that causes dead volumes and unphysiologic absorption, and no moving parts to fail. The platform can be highly predictive of human response and has a ratio of organ sizes, residence time of blood surrogate, removal of samples from blood surrogate, and the flow to each organ are physiologic.

When Hep G2/C3A cells were placed in each chamber all cell cultures retained high viability (85 to 95%) in a two day test. This test confirmed that this system provided sufficient delivery of oxygen and nutrients while emulating human physiology. Using a dye, flow pattern was as expected from the PBPK simulation; overall fluid turnover was physiologic. UH3 can populate a similar device with appropriate tissue mimics, electric connections, and fluid samplers. Other tissue mimics can be integrated with the current system design.

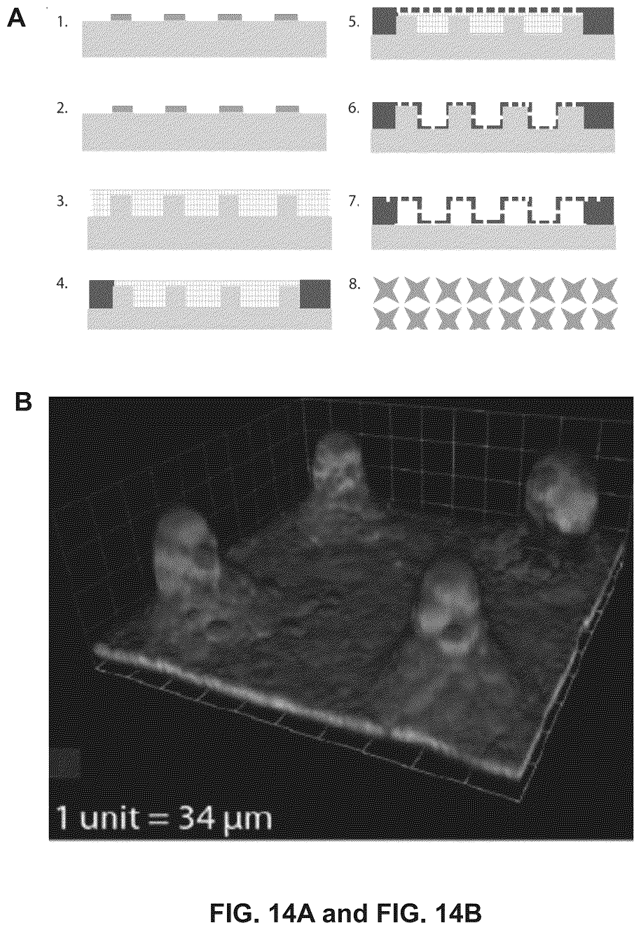

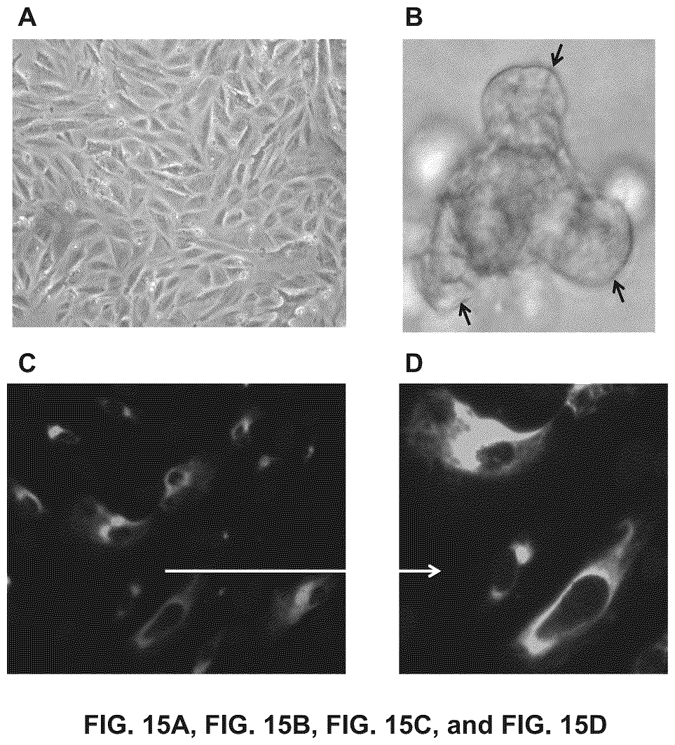

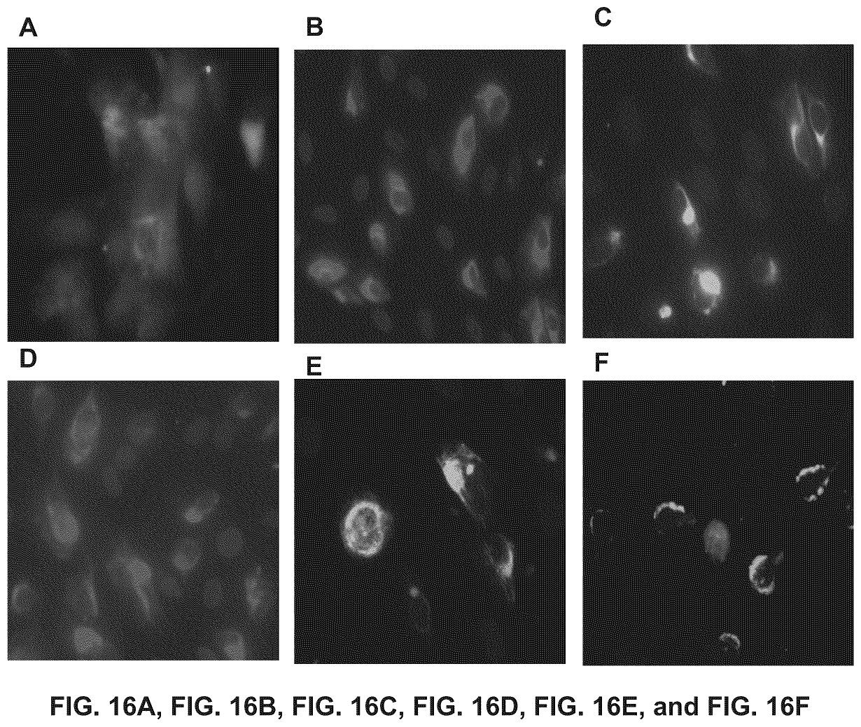

HepG2/C3A cell lines were used to demonstrate the ability to use the RegeneMed cell culture scaffold within the pumpless platform. Liver cells were co-cultivated with non-parenchymal cells for 14 days in dynamic conditions and showed a positive induction of CYP1A1 and CYP3A4 enzyme activity. The liver and the gastrointestinal tract can both be studied using the pumpless cell culture platform (see FIG. 13, FIG. 14B, FIG. 15A-FIG. 15D, and FIG. 16A-FIG. 16F).

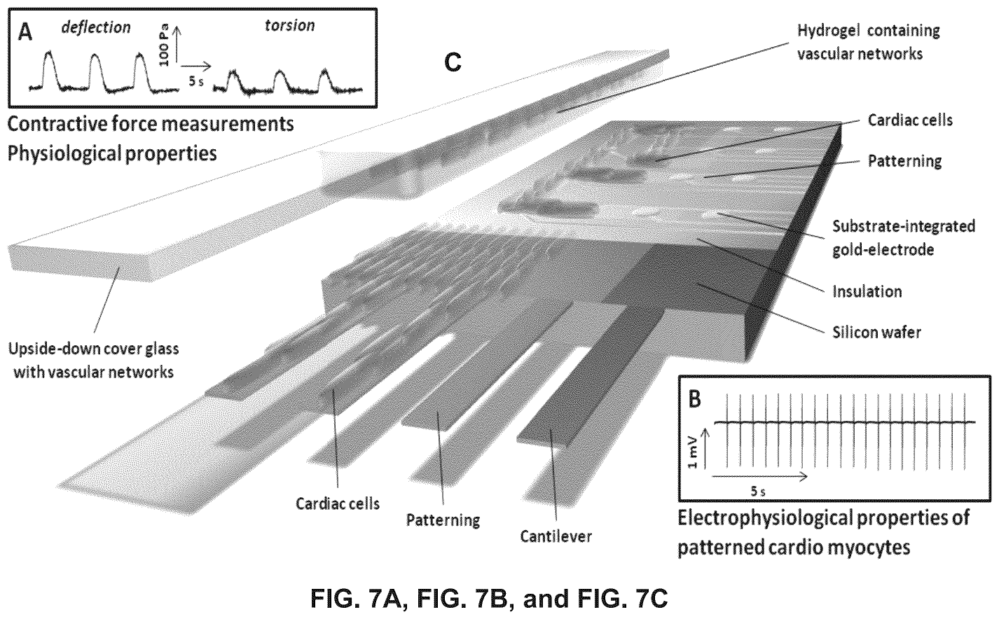

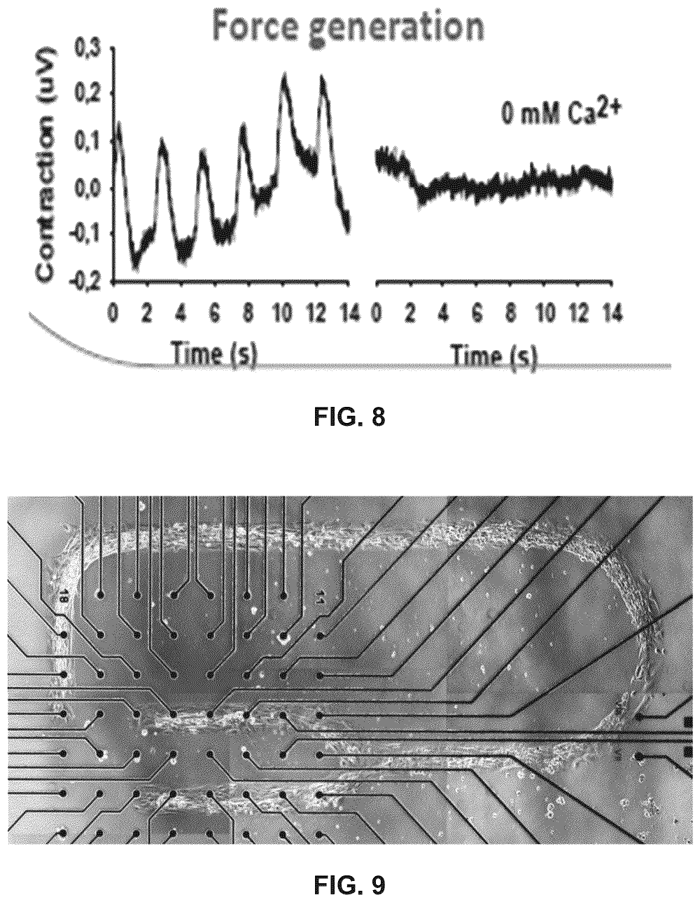

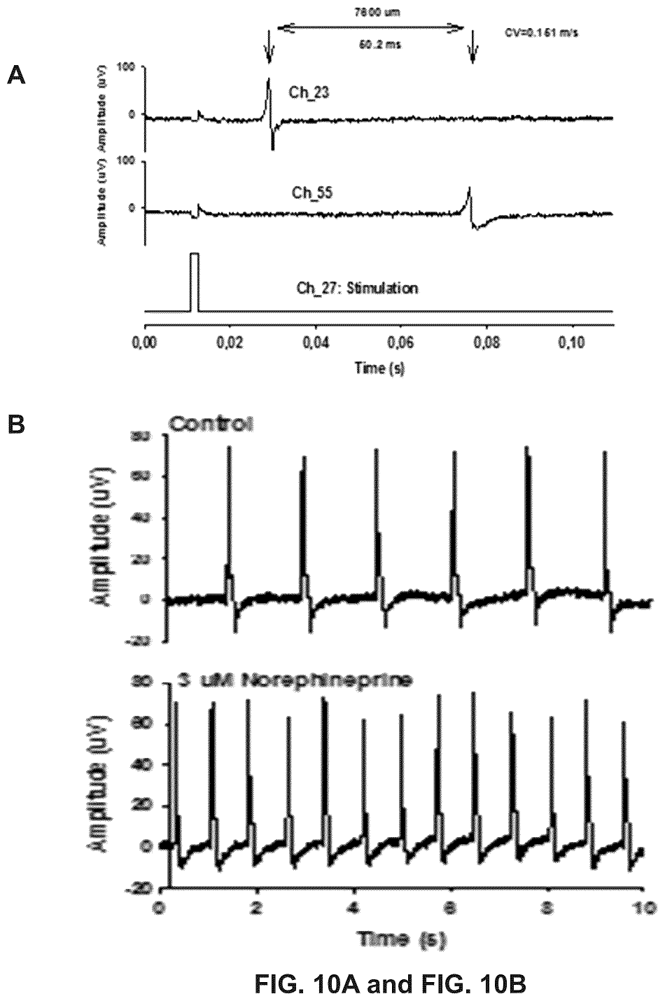

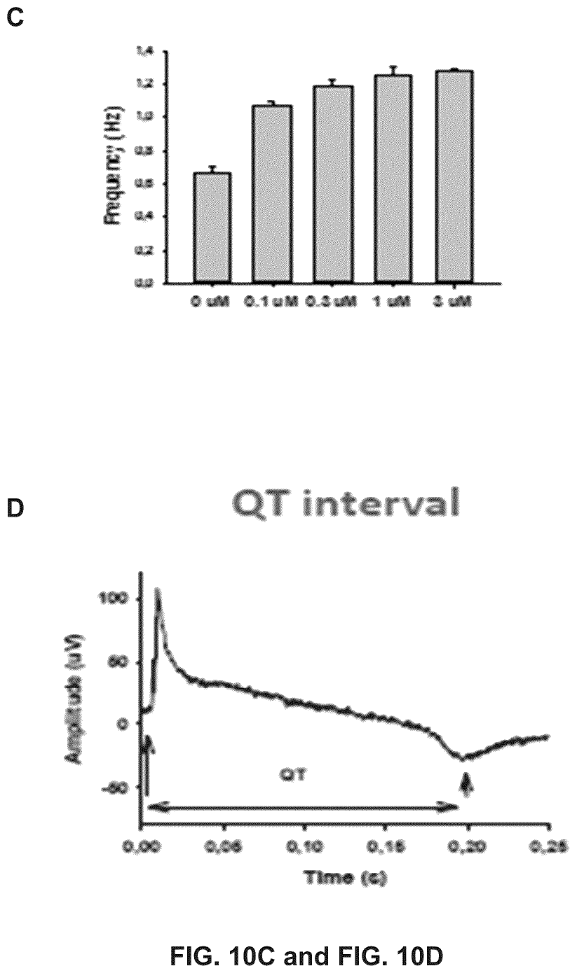

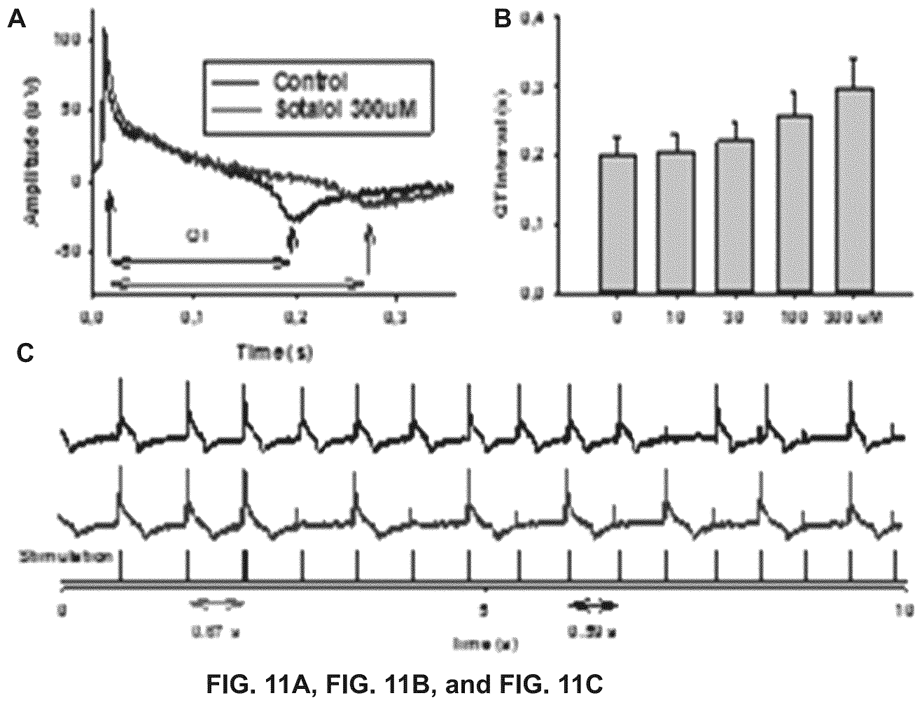

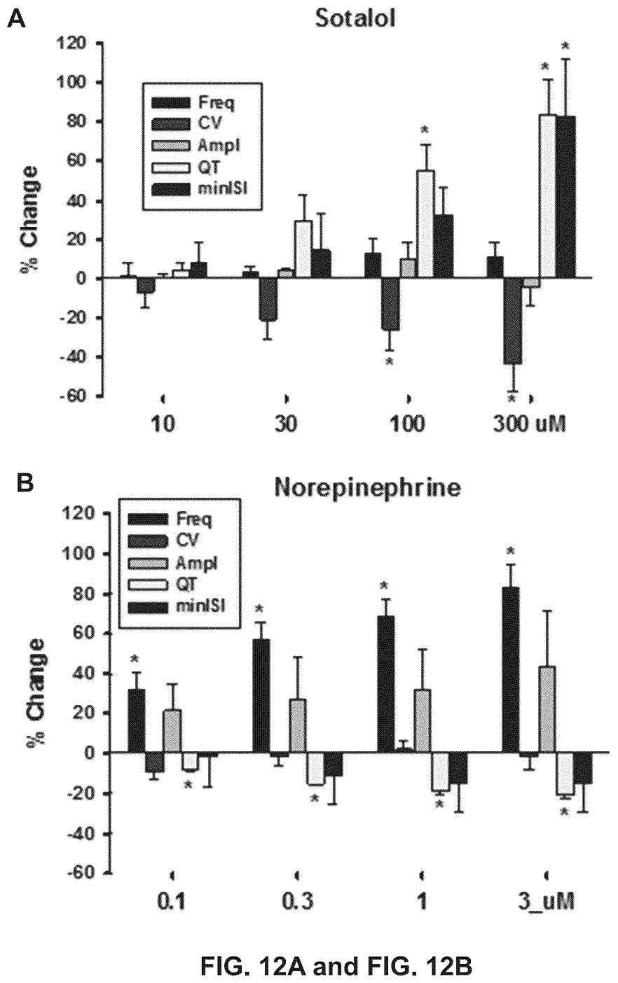

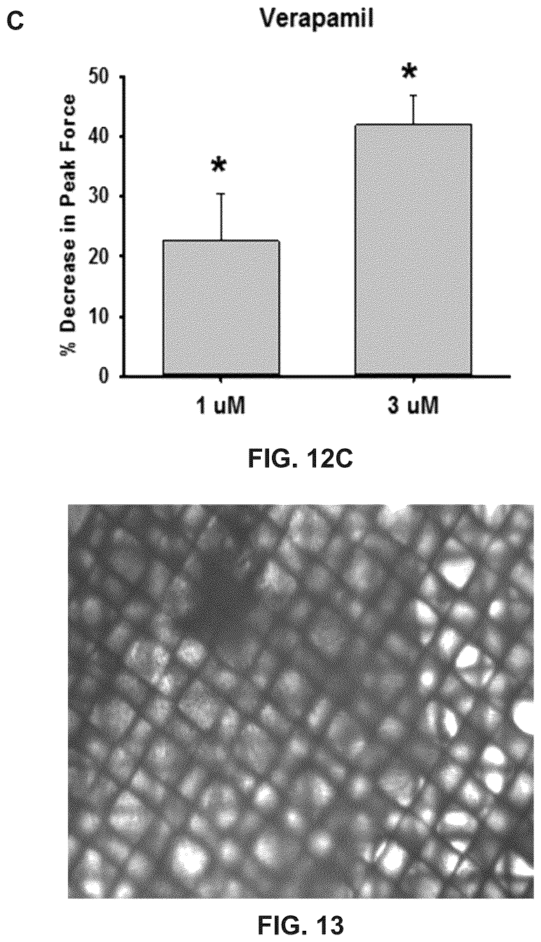

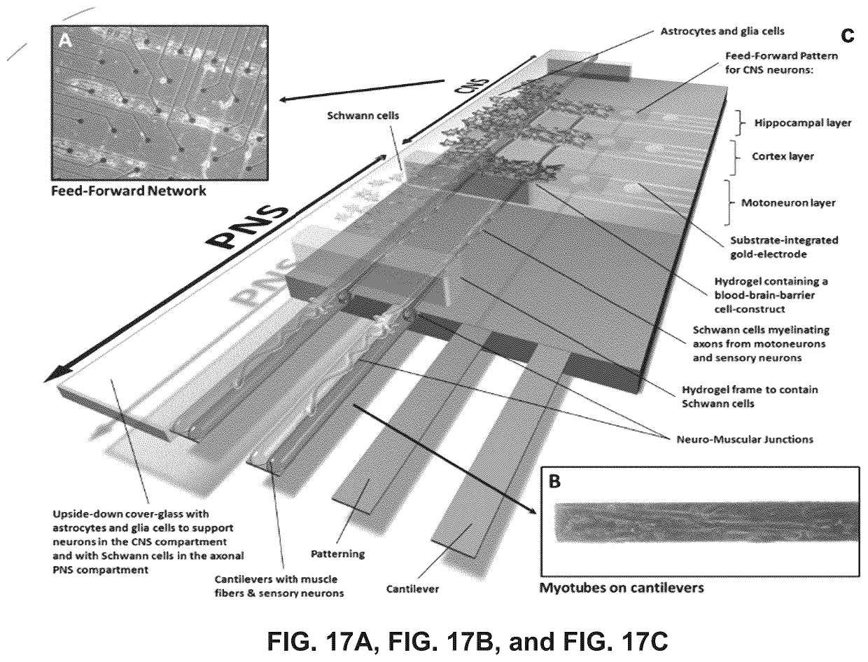

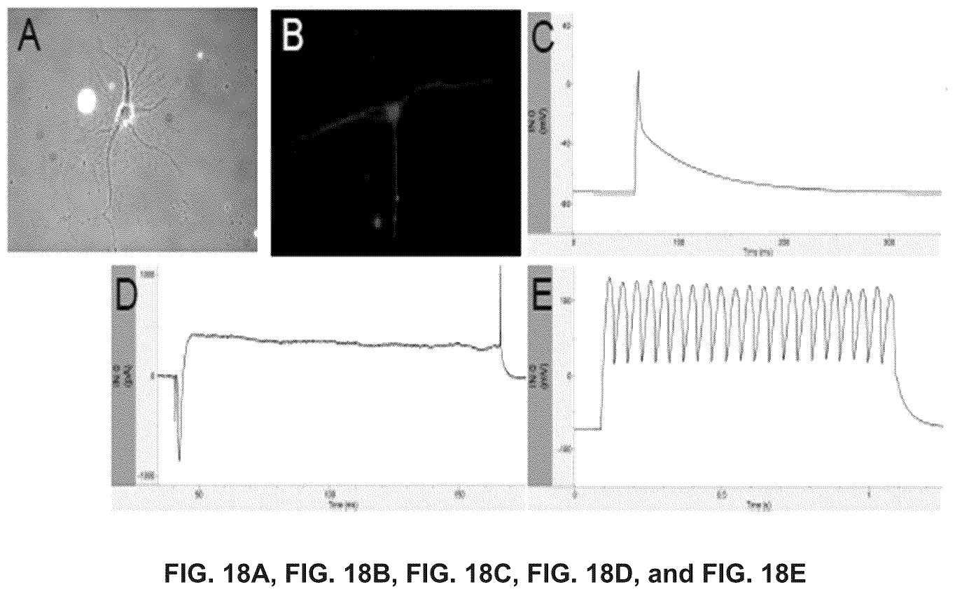

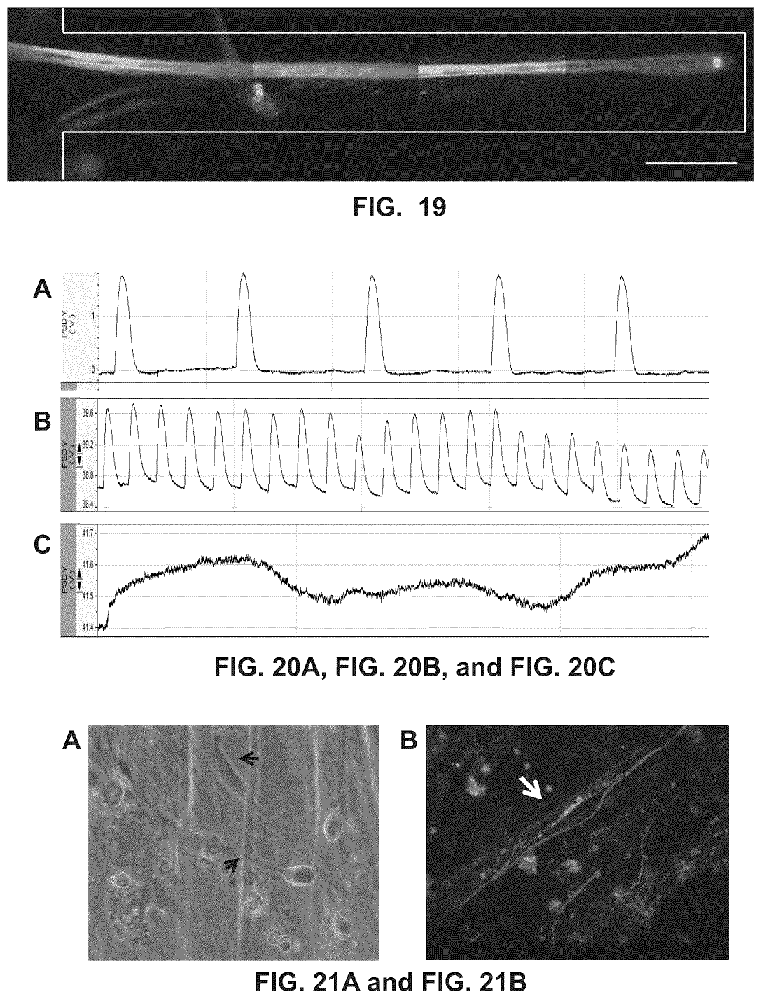

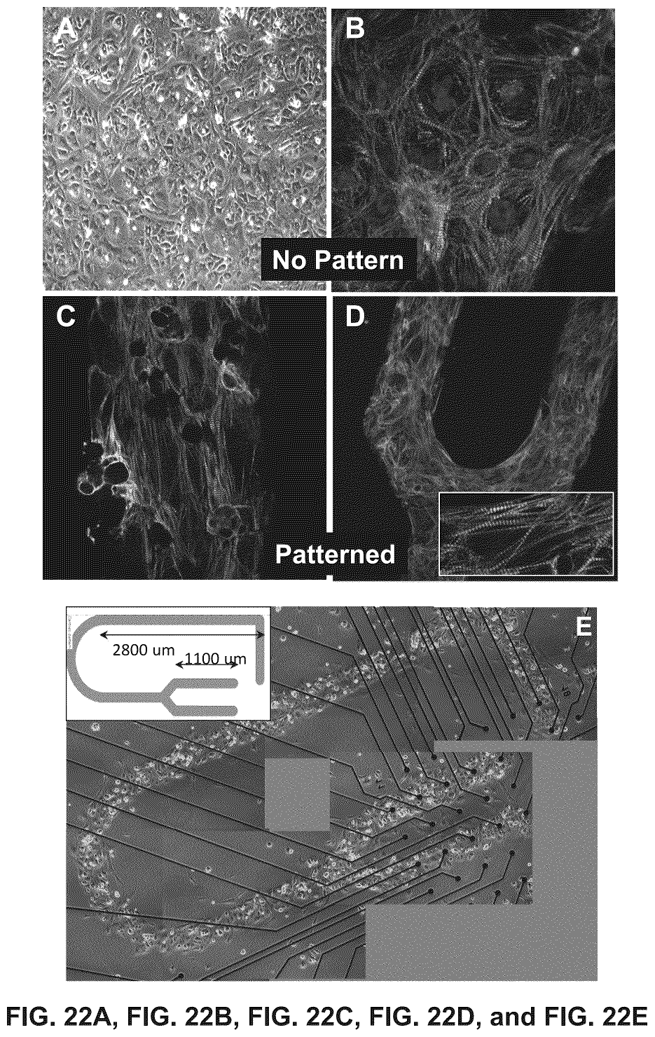

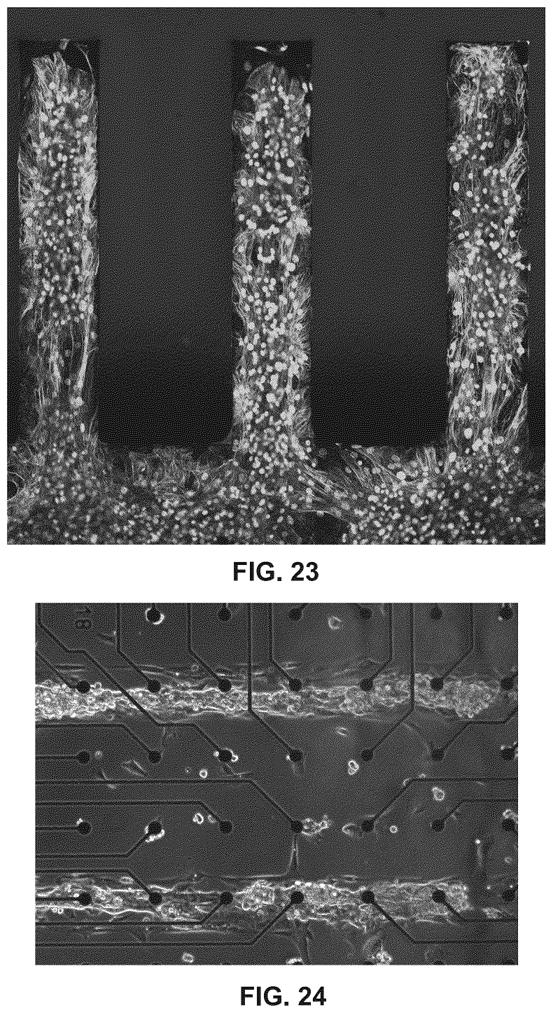

Use of the pumpless cell culture platform for the nervous system is shown in FIG. 17 and use of the pumpless cell culture platform for the cardiac system is shown in FIG. 7.

### iv) Experimental Advantages

An in vitro physiological system that represents cardiac function using a combination of chip based systems integrated with human stem cell derived cardiomyocytes was constructed and is described herein. A unique feature of the disclosed system is that the major determinants of human heart functions (e.g., rhythm generation, conduction, AP length, and force) were modeled by integrating 2-D cellular systems with silicon constructs. In other words, a potentially high-throughput 2-D cell culture-based integrated system that measures the major determinants of human cardiac function was generated. As electrical activity in the heart and cardiac muscle contraction are the primary focus of both pre-clinical toxicity and efficacy evaluation, the experiments provided herein deconstructed the heart into its primary functions and then reconstructed the data to predict drug effects in clinical trials.

To measure the relevant cardiac parameters, a platform was constructed using two devices: (1) extracellular surface-embedded microelectrodes for measuring the electrical properties of cardiac myocytes, and (2) AFM (Atomic Force Microscope) cantilevers for measuring the contractile force of the cardiac myocytes. The successful integration of cardiac myocytes into the force measurement platform described herein required several inventive steps including, but not limited to, the following: optimization of the serum-free medium for attachment and differentiation of human cardiac myocytes, adaptation and optimization of the surface-modification technique, adaptation of the force measurement and analysis system to cardiac myocytes, and validation of the system with selected reference compounds. Thus, the disclosed system provides a novel, functional high-throughput human cardiac myocyte-based screen for cardiac force generation.

The advantages of the disclosed systems are numerous: (1) the systems can be constructed in large scalable quantities, (2) the cells are organized by self-assembly without intervention, (3) the cells can be organized in a multiple well format, (4) data recording can be automated, and (5) drug administration can be automated. Furthermore, the use of human cardiac myocytes in the disclosed system eliminated the extrapolation problems generated from interspecies differences. These advantages, and others, converge to generate a high-throughput screening system that can measure numerous cardiac parameters including spontaneous beating frequency and force and velocity-force relationships.

Concerning the predictive value of the disclosed system: the measured in vitro electrophysiological parameters are analogous to the parameters used in the SCREENIT scoring system introduced by Hondeghem and coworkers in 1994 based on a rabbit model. In the disclosed system, beating frequency, conduction velocity, QT interval and peak force, reverse use dependence, variability in QT intervals and relative refractory period (which is related to triangulation) were all measured, which the predictive value of the screen.

Each of the following patent applications is incorporated herein by reference in its entirety: (1) U.S. patent application Ser. No. 12/661,323 filed on Mar. 15, 2000 and titled “Bio-Microelectromechanical System Transducer and Associated Methods”, (2) U.S. patent application Ser. No. 12/765,399 filed on Apr. 22, 2010 and titled “Method for Culturing Skeletal Muscle for Tissue Engineering”, (3) U.S. patent application Ser. No. 12/938,701 filed Nov. 3, 2010 and titled “Patterned Cardiomyocyte Culture on Microelectrode Array”, (4) U.S. patent application Ser. No13/102,672 filed on May 6, 2011 and titled “Formation of Neuromuscular Junctions in a Defined System”, (5) U.S. patent application Ser. No. 12/145,810 filed Jun. 25, 2008 and titled “Cell Culture Media and Process for Differentiation of Human Spinal Cord Stem Cells into Functional Motor Neuron Cells”, (6) U.S. patent application Ser. No 13/576,442 filed Feb. 7, 2011 and titled “Model and Methods for Identifying Points of Action in Electrically Active Cells”, (7) U.S. patent application Ser. No. 13/696,396 filed May 6, 2011 and titled “Formation of Neuromuscular Junctions”, (8) U.S. patent application Ser. No 12/117,339 filed May 8, 2008 and titled “Culture of Electrically Functional Adult Spinal Cord Neurons and Associated Methods”, (9) U.S. patent application Ser. No. 12/788,732 filed May 27, 2010 and titled “Method of Myelinating Isolated Motoneurons”, (10) U.S. patent application Ser. No 12/765,996 filed Apr. 23, 2010 and titled “Long Term In Vitro Culture of Tissue Engineered Functional Neuromuscular Junctions” (11) U.S. patent application Ser. No. 13/322,903 filed on May 28, 2010 and titled “In Vitro Production of Oligodendrocytes from Human Umbilical Cord Stem Cells”, (12) U.S. patent application Ser. No. 13/322,911 filed May 27, 2010 and titled “Method of Screening Drugs for Reversal of Amyloid Beta Neurotoxicity”, (13) U.S. Provisional Patent Application No. 61/684,168 filed Aug. 17, 2012 and titled “Methods, Systems and Compositions for In Vitro Cellular Models of Mammalian Systems”, (1 4) U.S. Provisional patent Application 61/758,628 filed Jan. 30, 2013 and titled “Compositions and Methods Comprising Cardiac Myocytes”, (15) U.S. Provisional Patent Application No. 61/732,042 filed Nov. 30, 2012 and titled “Derivation of Sensory Neurons and Neural Crest Stem Cells from Human Neural Progenitor HNP1”, (16) U.S. Provisional Patent Application No. 61/732,574 filed Dec. 3, 2012 and titled “Derivation of Sensory Neurons and Neural Crest Stem Cells from Human Neural Progenitor HNP1”, (17) U.S. Provisional Patent Application Ser. No. 6 1/7 84,923 filed Mar. 14, 2013 titled “Compositions and Methods for Generating Neural Crest Cells”, (18) U.S. Provisional Patent Application Ser. No. 61/78,9184, titled “Methods, Systems and Compositions for In Vitro Cellular Models of Mammalian Systems” filed Mar. 15, 2013, and (19) U.S. Provisional Patent Application Ser. No. 6 1/7 89,587, titled “Methods, Systems and Compositions for Concentric Cell Culture Analog Systems” filed Mar. 15, 2013, and applications concurrently filed herewith and each incorporated herein by reference in its entirety.

### H. References

Antzelevitch C. (2001) Transmural dispersion of repolarization and the T wave. Cardiovasc Res. 50: 426-431.

Antzelevitch C. (2005) Cardiac repolarization. The long and short of it. Europace. 7: 3-9.

Badie N, et al. (2009) A method to replicate the microstructure of heart tissue in vitro using DTMRI-based cell micropatterning. Ann Biomed Eng. 37: 2510-2521.

Belardinelli L, et al. (2003) Assessing predictors of drug-induced torsade de pointes. Trends Pharmacol Sci. 24: 619-625.

Bourgeois EB, et al. (2009) Change in conduction velocity due to fiber curvature in cultured neonatal rat ventricular myocytcs. IEEE Trans Biomed Eng. 56: 855-861.

Briggs M P, Practical Surface Analysis by Auger and X-ray Photoelectron Spectroscopy. New York, John Wiley and Sons (1992).

Campbell T J, et al. (2001) Therapeutic drug monitoring: antiarrhythmic drugs. Br J Clin Pharmacol. 52 Suppl 1: 21S-34S.

Carlsson L. (2006) In vitro and in vivo models for testing arrhythmogenesis in drugs. J Intern Med. 259: 70-80.

Choudhury A, et al. (2007) A piezoresistive microcantilever array for surface stress measurement: curvature model and fabrication. J Micromech Microeng. 17: 2065-2076.

Corey J M, et al. (1991) Compliance of hippocampal neurons to patterned substrate networks. J Neurosci Res. 30: 300-307.

Das M, et al. (2003) Electrophysiological and morphological characterization of rat embryonic motoneurons in a defined system. Biotechnol Prog. 19: 1756-1761.

Das M, et al. (2004) Long-term culture of embryonic rat cardiomyocytes on an organosilane surface in a serum-free medium. Biomaterials. 25: 5643-5647.

Das M, et al. (2005) Adult rat spinal cord culture on an organosilane surface in a novel serum-free medium. In Vitro Cell Dev Biol Anim. 41: 343-348.

Das M, et al. (2006) A defined system to allow skeletal muscle differentiation and subsequent integration with silicon microstructures. Biomaterials. 27: 4374-4380.

Das M, et al. (2007) Auto-catalytic ceria nanoparticles offer neuroprotection to adult rat spinal cord neurons. Biomaterials. 28: 1918-1925.

Das M, et al. (2007) Differentiation of skeletal muscle and integration of myotubes with silicon microstructures using serum-free medium and a synthetic silanc substrate. Nat Protoc. 2: 1795-1801.

Das M, et al. (2007) Embryonic motoneuron-skeletal muscle co-culture in a defined system. Neuroscience. 146: 481-488.

Das M, et al. (2008) Temporal neurotransmitter conditioning restores the functional activity of adult spinal cord neurons in long-term culture. Exp Neurol. 209: 171-180.

Das M, et al. (2009) Developing a novel serum-free cell culture model of skeletal muscle differentiation by systematically studying the role of different growth factors in myotube formation. In Vitro Cell Dev Biol Anim. 45: 378-387.

Das M, et al. (2009) Skeletal Muscle Tissue Engineering: An Improved Model Promoting Long Term Survival of Myotubes, Structural Development of E-C Coupling Apparatus and Neonatal Myosin Heavy Chain (MHC) Expression. Biomaterials. 30: 5392-5402.

Das M, et al. (2010) A defined long-term in vitro tissue engineered model of neuromuscular junctions. Biomaterials. 31: 4880-4888.Datar et al. MRS Bulletin. 34, 449 (2009).

De Clerck F, et al. (2002) In vivo measurement of QT prolongation, dispersion and arrhythmogenesis: application to the preclinical cardiovascular safety pharmacology of a new chemical entity. Fundam Clin Pharmacol. 16: 125-140.

Dhir V, et al. (2009) Patterning of diverse mammalian cell types in serum free medium with photoablation. Biotechnol Prog. 25: 594-603.

Edwards D, et al. (2010) Addition of glutamate to serum-free culture promotes recovery of electrical activity in adult hippocampal neurons in vitro. J Neurosci Methods. 190: 155-163

Guo X, et al. (2011) Neuromuscular junction formation between human stem cell-derived motoneurons and human skeletal muscle in a defined system. Biomaterials. 32: 9602-9611.

Guo X F, et al. (2010) Neuromuscular junction formation between human stem-cell-derived motoneurons and rat skeletal muscle in a defined system. Tissue Eng Part C Methods. 16: 1347-1355.

Hondeghem L M and Hoffman P. (2003) Blinded test in isolated female rabbit heart reliably identifies action potential duration prolongation and proarrhythmic drugs: importance of triangulation, reverse use dependence, and instability. J Cardiovasc Pharmacol. 41: 14-24.

Hondeghem L M, et al. (2001) Instability and triangulation of the action potential predict serious proarrhythmia, but action potential duration prolongation is antiarrhythmic. Circulation. 103: 2004-2013.

Hondeghem L M, et al. (2003) Detection of proarrhythmia in the female rabbit heart: blinded validation. J Cardiovasc Electrophysiol. 14: 287-294.

Hondeghem L M. (2006) Thorough QT/QTc not so thorough: removes torsadogenic predictors from the T-wave, incriminates safe drugs, and misses profibrillatory drugs. J Cardiovasc Electrophysiol. 17: 337-340.

Hondeghem L M. (2007) Relative contributions of TRIaD and QT to proarrhythmia. J Cardiovasc Electrophysiol. 18: 655-657. Jackson JH 4th, et al. (2004) Assessment of drug therapy management and the prevalence of heart failure in a managed care population with hypertension. J Manag Care Pharm. 10: 513-520.

King T, et al. (2000) Piezoactuators for ‘real-world’ applications—Can they deliver sufficient displacement? Power Engineering. 14: 105-110.“\.

Lawrence C L, et al. (2005) Nonclinical proarrhythmia models: predicting Torsades de Pointes. J Pharmacol Toxicol Methods. 52: 46-59.

Lawrence C L, et al. (2006) A rabbit Langendorff heart proarrhythmia model: predictive value for clinical identification of Torsades de Pointes. Br J Pharmacol. 149: 845-860.

Lin J W, et al. (2008) Region [corrected] of slowed conduction acts as core for spiral wave reentry in cardiac cell monolayers. Am J Physiol Heart Circ Physiol. 294: H58-H65.

Liu T X, et al. (2006) Blinded validation of the isolated arterially perfused rabbit ventricular wedge in preclinical assessment of drug-induced proarrhythmias. Heart Rhythm. 3: 948-956.

Lochter A J, et al. (1995) Control of neuronal morphology in vitro: interplay between adhesive substrate forces and molecular instruction. J Neurosci Res. 42: 145-158.

Lou X J. (2009) Polarization fatigue in ferroelectric thin films and related materials. Journal of Applied Physics. 105: 024101-024124.

Moe G K. (1962) On the multiple wavelet hypothesis of atrial fibrillation. Arch Int Pharmacodyn Ther. 183-188.

Molnar P, et al. (2007) Photolithographic Patterning of C2C12 Myotubes using Vitronectin as Growth Substrate in Serum-Free Medium. Biotechnol Prog. 23: 265-268.

Mutyala M S K, et al. (2009) Mechanical and electronic approaches to improve the sensitivity of microcantilever sensors. Acta Mechanica Sinica. 25: 1-12.

Nash M P, et al. (2006) Evidence for multiple mechanisms in human ventricular fibrillation. Circulation. 114: 536-542.

Natarajan A, et al. (2006) Microelectrode array recordings of cardiac action potentials as a high throughput method to evaluate pesticide toxicity. Toxicol In Vitro. 20: 375-381.

Natarajan A, et al. (2008) Growth and electrophysiological properties of rat embryonic cardiomyocytes on hydroxyl- and carboxyl-modified surfaces. J Biomater Sci Polym Ed. 19: 1319-1331.

Natarajan A, et al. (2011) Patterned cardiomyocytes on microelectrode arrays as a functional, high information content drug screening platform. Biomaterials. 32: 4267-4274.

Nguemo F, et al. (2012) In vitro model for assessing arrhythmogenic properties of drugs based on high-resolution impedance measurements. Cell Physiol Biochem. 29: 819-832.

Parker K K, et al. (2008) Myofibrillar architecture in engineered cardiac myocytes. Circ Res. 103: 340-342.

Peng H B, et al. (2003) Differential effects of neurotrophins and schwann cell-derived signals on neuronal survival/growth and synaptogenesis. J Neurosci. 23: 5050-5060.

Pijnappels D A, et al. (2007) Resynchronization of separated rat cardiomyocyte fields with genetically modified human ventricular scar fibroblasts. Circulation. 116: 2018-2028.

Popat K C, et al. (2004) Surface modification of nanoporous alumina surfaces with poly(ethylene glycol). Langmuir. 20: 8035-8041.

Popat K C, et al. (2004) Quantitative xps analysis of peg-modified silicon surfaces. J Phys Chem. 108: 5185-5188.Ravenscroft MS, et al. J Am Chem Soc. 120, 12169 (1998)

Recanatini M, et al. (2005) QT prolongation through hERG K(+) channel blockade: current knowledge and strategies for the early prediction during drug development. Med Res Rev. 25: 133-166.

Rumsey J W, et al. (2008) Tissue Engineering Intrafusal Fibers: Dose and Time Dependent Differentiation of Nuclear Bag Fibers in a Defined In Vitro System using Neuregulin 1- beta-1. Biomaterials. 29: 994-1004.

Rumsey J W, et al. (2009) Node of Ranvier formation on motoneurons in vitro. Biomaterials. 30: 3567-3572.

Rumsey J W, et al. (2010) Tissue engineering the mechanosensory circuit of the stretch reflex arc: sensory neuron innervation of intrafusal muscle fibers. Biomaterials. 31: 8218-8227. Sala M, et al. Ther Adv Cardiovasc Dis. 3, 29 (2009).

Sathayc A, et al. (2006) Electrical pacing counteracts intrinsic shortening of action potential duration of neonatal rat ventricular cells in culture. J Mol Cell Cardiol. 41: 633-641.

Suter W. (2006) Predictive value of in vitro safety studies. Curr Opin Chem Biol. 10: 362-366.

Valentin J P, et al. (2004) Review of the predictive value of the Langendorff heart model (Screenit system) in assessing the proarrhythmic potential of drugs. J Pharmacol Toxicol Methods. 49: 171-181.

Varghese K, et al. (2009) Regeneration and characterization of adult mouse hippocampal neurons in a defined in vitro system. J Neurosci Methods. 177: 51-59.

Varghese K, et al. (2010) A new target for amyloid beta toxicity validated by standard and high-throughput electrophysiology. PLoS One. 5: e8643.

Wilson K, et al. (2010) Measurement of contractile stress generated by cultured rat muscle on silicon cantilevers for toxin detection and muscle performance enhancement. PLoS One. 5: e11042.

1-35. (canceled)

36. A system for measuring contractile properties and electrical properties of a cardiac cell culture, the system comprising:
a microelectrode array configured to detect an electrical property from cells cultured thereon;
a plurality of cantilevers configured to detect contractile forces from cells cultured thereon;
a monolayer of cardiac myocytes cultured on the microelectrode array and the plurality of cantilevers; and
a computing device configured to:
receive an electrical property reading from the monolayer via the microelectrode array;
receive a contractile force reading from the monolayer via the plurality of cantilevers;
determine multiple cardiac parameters based on the electrical property reading and the contractile force reading;
measure a change in each of the multiple cardiac parameters in response to an agent that contacts the monolayer;
create a fingerprint for the agent based on the change in each of the multiple cardiac parameters.

37. The system of claim 36, wherein the plurality of cantilevers comprises piezoelectric or piezoresistive materials.

38. The system of claim 36, wherein the monolayer of cardiac myocytes is patterned onto the microelectrode array and the plurality of cantilevers.

39. The system of claim 36, wherein a surface of the plurality of cantilevers comprises a (3-Trimethoxysilylpropyl) diethylenetriamine coating.

40. The system of claim 36, wherein a surface of the plurality of cantilevers comprises a fibronectin coating.

41. The system of claim 36, wherein the microelectrode array comprises glass.

42. The system of claim 36, wherein a surface of the microelectrode array comprises adsorbed proteins.

43. The system of claim 36, wherein a surface of the microelectrode array comprises self-assembled monolayers (SAMs).

44. The system of claim 43, wherein the SAMs comprise extracellular matrix components.

45. The system of claim 36, further comprising a medium contacting the monolayer of cardiac myocytes.

46. The system of claim 45, wherein the medium comprises epidermal growth factor (EGF).

47. The system of claim 45, wherein the medium comprises hydrocortizone and L-thyroxin.

48. The system of claim 45, wherein the medium comprises the agent that contacts the monolayer.

49. The system of claim 48, wherein the agent is a metabolic inhibitor, a nutritional supplement, a therapeutic compound, composition, and drug, an investigational compound, composition, and drug, a biosimilar, an agonist, an antagonist, a hormone, a growth factor, a small molecule, a monoclonal antibody, and combinations thereof.

50. The system of claim 36, wherein at least one of the multiple cardiac parameters is a conduction velocity.

51. The system of claim 36, wherein at least one of the multiple cardiac parameters is a peak contractile force.

52. The system of claim 36, wherein the multiple cardiac parameters comprise one or more of: spontaneous beating rate, conduction velocity, QT interval, minimal interspike interval, peak contractile force, speed of contraction, time to relaxation, an arrhythmogenic mechanism, and field potential amplitude.

53. The system of claim 36, wherein the computing device is further configured to predict cardiac side effects of the agent.

54. The system of claim 36, wherein the fingerprint comprises information on how the agent affects the multiple cardiac parameters in a way that is characteristic of the agent.

55. The system of claim 54, wherein the fingerprint reflects the mechanism of action of the agent and the physiological effects of the agent.
